SEQUENCE LISTING (19 sequences)

SEQ ID NO: 1 - protein, 213 aa, Homo sapiens
MQLPLALCLVCLLVHTAFRVVEGQGWQAFKNDATEIIPELGEYPEPPPELENNKTMNRAENGGRPPHHPFETKDVSEYSCRELHFTRYVTDGPCRSAKPVTELVCSGQCGPARLLPNAIGRGKWWRPSGPDFRCIPDRYRAQRVQLLCPGGEAPRARKVRLVASCKCKRLTRFHNQSELKDFGTEAARPQKGRKPRPRARSAKANQAELENAY

SEQ ID NO: 2 - protein, 10 aa, Homo sapiens
GFTFRSHWLS

SEQ ID NO: 3 - protein, 20 aa, Homo sapiens
WVSNINYDGSSTYYADSVKG

SEQ ID NO: 4 - protein, 8 aa, Homo sapiens
DTYLHFDY

SEQ ID NO: 5 - protein, 14 aa, Homo sapiens
TGTSSDVGDINDVS

SEQ ID NO: 6 - protein, 11 aa, Homo sapiens
LMIYDVNNRPS

SEQ ID NO: 7 - protein, 10 aa, Homo sapiens
QSYAGSYLSE

SEQ ID NO: 8 - protein, 117 aa, Homo sapiens
QVQLVESGGGLVQPGGSLRLSCAASGFTFRSHWLSWVRQAPGKGLEWVSNINYDGSSTYYADSVKGRFTISRDNSKNTLYLQMNSLRAEDTAVYYCARDTYLHFDYWGQGTLVTVSS

SEQ ID NO: 9 - protein, 113 aa, Homo sapiens
DIALTQPASVSGSPGQSITISCTGTSSDVGDINDVSWYQQHPGKAPKLMIYDVNNRPSGVSNRFSGSKSGNTASLTISGLQAEDEADYYCQSYAGSYLSEVFGGGTKLTVLGQ

SEQ ID NO: 10 - protein, 443 aa, Homo sapiens
QVQLVESGGGLVQPGGSLRLSCAASGFTFRSHWLSWVRQAPGKGLEWVSNINYDGSSTYYADSVKGRFTISRDNSKNTLYLQMNSLRAEDTAVYYCARDTYLHFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 11 - protein, 217 aa, Artificial Sequence
  Homo sapiens
DIALTQPASVSGSPGQSITISCTGTSSDVGDINDVSWYQQHPGKAPKLMIYDVNNRPSGVSNRFSGSKSGNTASLTISGLQAEDEADYYCQSYAGSYLSEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 12 - protein, 449 aa, Homo sapiens
EVQLVQSGAEVKKPGASVKVSCKASGYTFTDYNMHWVRQAPGQGLEWMGEINPNSGGAGYNQKFKGRVTMTTDTSTSTAYMELRSLRSDDTAVYYCARLGYDDIYDDWYFDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 13 - protein, 214 aa, Homo sapiens
DIQMTQSPSSLSASVGDRVTITCRASQDISNYLNWYQQKPGKAPKLLIYYTSRLLSGVPSRFSGSGSGTDFTLTISSLQPEDFATYYCQQGDTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 444 aa, Homo sapiens
QVQLVQSGAEVKKPGASVKVSCKVSGFPIKDTFQHWVRQAPGKGLEWMGWSDPEIGDTEYASKFQGRVTMTEDTSTDTAYMELSSLRSEDTAVYYCATGDTTYKFDFWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 15 - protein, 214 aa, H sapiens
DIQMTQSPSSLSASVGDRVTITCKASQDVHTAVAWYQQKPGKAPKLLIYWASTRWTGVPSRFSGSGSGTDFTLTISSLQPEDFATYYCQQYSDYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 26 aa, Homo sapiens
CGPARLLPNAIGRGKWWRPSGPDFRC

SEQ ID NO: 17 - protein, 60 aa, Artificial Sequence
  Homo sapiens
DVSEYCRELHFTRSAKPVTELVCSGQCGPARWWRPSGPDFRCIPDRYRLVASCKCKRLTR

SEQ ID NO: 18 - DNA, 20 bp, Artificial Sequence
  Forward primer
ccacggaaatcatccccgag

SEQ ID NO: 19 - DNA, 20 bp, Artificial Sequence
  Reverse primer
catcggtcacgtagcgggtg

(19) United States
(12) Patent Application Publication
(10) Pub. No.: US 2021/0198350 A1
(43) Pub. Date: Jul. 1, 2021

(54) USE OF A SCLEROSTIN ANTAGONIST

(71) Applicant: MEREO BIOPHARMA 3 LIMITED, London (GB)

(72) Inventors: Eric HESSE, Hamburg (DE); Hanna TAIPALEENMAKI, Hamburg (DE)

(73) Assignee: MEREO BIOPHARMA 3 LIMITED, London (GB)

(21) Appl. No.: 17/256,572

(22) PCT Filed: Jun. 28, 2019
(86) PCT No.: PCT/EP2019/067467

§ 371 Date: Dec. 28, 2020

(30) Foreign Application Priority Data
Jun. 29, 2018 (GB) 1810746.6

(51) Int. Cl. C07K 16/22; A61K 45/06

(52) U.S. Cl. CPC C07K 16/22; A61K 2039/505; A61K 45/06

(57) ABSTRACT

The present invention relates to methods and compositions for treating a myopathy in a subject, comprising administering a therapeutically effective amount of a sclerostin antagonist to the subject. The myopathy may be characterized by a loss of skeletal muscle mass, size, strength and/or function. The sclerostin antagonist may be an anti-sclerostin antibody.

### TECHNICAL FIELD

This invention is in the field of treating a myopathy, and in particular a myopathy characterized by a loss of skeletal muscle mass, size, strength and/or function.

### BACKGROUND

Myopathies are diseases or disorders of skeletal muscle tissue or muscles. Primary symptoms include muscle weakness due to dysfunction of muscle fiber as well as muscle atrophy. Cramps, myalgias, and exertional fatigue are other common presenting symptoms. Cancer-associated myopathies include cancer cachexia, which leads to progressive functional impairment, treatment-related complications, poor quality of life and cancer-related mortality. By some estimates, nearly one-third of cancer deaths can be attributed to cancer cachexia. Current treatments for myopathies include drug therapy and physical therapy.

There remains a need for further and improved treatments for myopathies, and it is an object of the invention to provide these.

### DISCLOSURE OF THE INVENTION

### Methods of Treatment

The invention provides a method of treating a myopathy in a subject, comprising administering a therapeutically effective amount of a sclerostin antagonist to the subject.

A myopathy is a disease or disorder of skeletal muscle tissue or muscles. Primary symptoms include muscle weakness due to dysfunction of muscle fiber as well as muscle atrophy. Cramps, myalgias, and exertional fatigue are other common presenting symptoms. Diagnosis of a myopathy is routine for those skilled in the art and may be based on the medical history and physical examination tests including the chair rising test, timed up-and-go test, tandem stand test and hand grip test. Diagnostic tests may include a blood test to measure potassium levels and the level of various muscle enzymes (e.g. creatine kinase (CK), lactic acid dehydrogenase (LDH) and pyruvate kinase (PK)) and myositis-specific antibodies, an electromyogram (EMG) to gauge electrical activity in muscle, muscle biopsy, computerised tomography (CT), magnetic resonance imaging (MRI), ultrasound or bioelectrical analysis to identify abnormal muscle, muscle loss and fatty degeneration, and genetic testing.

In one embodiment of the invention, the myopathy is characterized by a loss of skeletal muscle mass, size, strength and/or function. As noted above, these parameters can be routinely determined by those skilled in the art. A loss of skeletal muscle mass and/or size can be determined by one or more of CT, MRI, ultrasound or biopsy, for example. A loss of skeletal muscle strength and/or function can be determined by one or more of chair rising test, tandem stand test, timed up-and-go test, handgrip test, EMG, and diagnostic blood tests, for example. A loss of skeletal muscle function can also be determined by one or more of chair rising test, tandem stand test, timed up-and-go test, handgrip test, EMG, and diagnostic blood tests, for example. In one embodiment, the myopathy is characterized by a loss of skeletal muscle mass. In one embodiment, the myopathy is characterized by a loss of skeletal muscle size. In one embodiment, the myopathy is characterized by a loss of skeletal muscle strength. In one embodiment, the myopathy is characterized by a loss of skeletal muscle function. Examples of myopathies characterized by a loss of skeletal muscle mass, size, strength and/or function include cachexia, sarcopenia, muscular dystrophies (MD) such as Duchenne muscular dystrophy (DMD) or Becker muscular dystrophy (DMB). Thus, in one embodiment the myopathy is cachexia. In one embodiment, the myopathy is sarcopenia. In one embodiment the myopathy is a muscular dystrophy (MD), such as DMD or BMD. Skeletal muscle mass, size, strength and/or function can also be lost due to immobilization, loss of gravity or disuse.

In one embodiment the myopathy is a cancer-associated loss of skeletal muscle mass, size, strength and/or function. Examples of cancer-associated loss of skeletal muscle mass, size, strength and/or function include cachexia and cancer-associated myositis (CAM), inflammatory myopathy and steroid-induced loss of skeletal muscle mass, size, strength and/or function. The cancer-associated loss of skeletal muscle mass, size, strength and/or function may be due to breast cancer, lung cancer, prostate cancer, multiple myelcoma, cholangiocarcinoma, or hepatocellular carcinoma. In one embodiment the cancer is breast cancer. Thus in one embodiment, the invention provides a method of treating a breast-cancer associated loss of skeletal muscle mass, size, strength and/or function in a subject, comprising administering a therapeutically effective amount of a sclerostin antagonist to the subject.

In one embodiment, the myopathy is further characterized by an increased level of sclerostin expression. That is, the patient suffering from the myopathy has an increased level of sclerostin expression relative to patients not suffering from the myopathy. Methods for determining sclerostin expression and concentrations in biological samples are routine for those skilled in the art (see e.g. Bezooijen et al. 2004, J Exp Med Vol. 199(6):805-814 and McNulty et al. 2011, JCEM Vol. 96(7):E1159-E1162, which methods are expressly incorporated herein by reference thereto).

The inventors believe that the use of a sclerostin antagonist according to the invention is useful to increase skeletal muscle mass, size, strength and/or function in a patient suffering from a myopathy, in particular in the context of patient suffering from a cancer-associated loss of skeletal muscle mass, size, strength and/or function such as cachexia. Treatment with a sclerostin antagonist may also prolong survival/increase lifespan of the patient suffering from a myopathy. Thus, in one embodiment administration of the therapeutically effective amount of the sclerostin antagonist increases skeletal muscle mass, size, strength and/or function. In one embodiment, administration of the therapeutically effective amount of the sclerostin antagonist increases skeletal muscle mass. In one embodiment administration of the therapeutically effective amount of the sclerostin antagonist increases skeletal muscle size. In one embodiment administration of the therapeutically effective amount of the sclerostin antagonist increases skeletal muscle strength. In one embodiment administration of the therapeutically effective amount of the sclerostin antagonist increases skeletal muscle function.

In one embodiment, the method of the invention comprises administering to the subject a therapeutically effective amount of an additional therapeutic agent, such as an anti-cancer drug and/or an agent for the treatment of a myopathy. Anti-cancer drugs and agents for the treatment of a myopathy are well known to those skilled in the art. Non-exhaustive examples of anti-cancer drugs include chemotherapy agents, such as imatinib, lenalidomide, bortezomib, leuprorelin, abiraterone and pemetrexed and monoclonal antibodies such as rituximab, bevacizumab, trastuzunnab, and cetuximab. Examples of agents for the treatment of a myopathy include bone sparing drugs such as bisphosphonates, zoledronic acid, denosumab, alendronate, etidronate, ibandronate, risedronate and, teriparatide, abaloparatide and calcitriol. The sclerostin antagonist of the invention and the additional therapeutic agent may be administered simultaneously, separately or sequentially.

In another aspect, the disclosure provides a method of treating cancer in a subject, comprising administering a therapeutically effective amount of a sclerostin antagonist to the subject. All of the embodiments described herein in relation to the treatment of a myopathy apply equally to the treatment of cancer aspect of the disclosure. Thus, and by way of example only, in one embodiment the cancer is breast cancer and in one embodiment the sclerostin antagonist is the anti-sclerostin antibody setrusumab.

Cancer-induced osteolytic disease is defined as the dissemination of cancer cells into the bone marrow, with accompanying osteolytic destruction and/or osteoblastic destruction. Cancer-induced osteolytic disease causes pain, spinal cord compression and increased risk of fractures. Cancer-induced osteolytic disease is associated with breast, prostate, lung renal, thyroid, skin, ovarian cancers as well as multiple myeloma. Diagnosis of cancer-induced osteolytic disease is routine for those skilled in the art and may be based on various imaging modalities including radiography, magnetic resonance imaging (MRI), computed tomography (CT) and 18FDG-PET.

In another aspect, the disclosure provides a method of treating cancer-induced osteolytic disease in a subject, comprising administering a therapeutically effective amount of a sclerostin antagonist to the subject. All of the embodiments described herein in relation to the treatment of a myopathy apply equally to the treatment of a cancer-induced osteolytic disease aspect of the disclosure. Thus, and by way of example only, in one embodiment the cancer is breast cancer and in one embodiment the sclerostin antagonist is the anti-sclerostin antibody setrusumab.

As used herein, the terms “subject” or “individual” or “patient” refers to someone in need of therapy. As used herein, the term “subject” includes any human or nonhuman animal. The term “nonhuman animal” includes all vertebrates, e.g. mammals and non-mammals, such as mice, rats, nonhuman primates, sheep, dogs, cats, horses and cows. Typically, however, the term “subject” refers to a human.

The terms “effective amount” or “amount effective to” or “therapeutically effective amount” includes reference to a dosage of a therapeutic agent sufficient to produce a desired result, in particular the prevention of disease progression and/or the amelioration of symptoms associated with the disease for which the subject is being treated.

As used herein, the terms “treat”, “treating” or “treatment” refer to therapeutic measures, wherein the object is to prevent or slow down (lessen) an undesired physiological change or disorder, such as the loss of skeletal muscle mass, size, strength and/or function. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total). “Treatment” can also mean prolonging survival as compared to expected survival if not receiving treatment. A subject in need of treatment typically refers to a patient who is already suffering from the disease, condition or disorder for which treatment is provided.

In one aspect, the disclosure provides a sclerostin antagonist for use in treating a myopathy in a subject. All of the embodiments/disclosures described herein in relation to the methods of treatment of a myopathy apply equally to this medical use aspect of the disclosure.

In one aspect, the disclosure provides the use of a sclerostin antagonist in the manufacture of a medicament to treat a myopathy. All of the embodiments/disclosures described herein in relation to the methods of treatment of a myopathy apply equally to this use aspect of the disclosure.

In one aspect, the disclosure provides a sclerostin antagonist for use in treating cancer in a subject. All of the embodiments/disclosures described herein in relation to the methods of treatment of cancer in a subject apply equally to this medical use aspect of the disclosure.

In one aspect, the disclosure provides a sclerostin antagonist for use in treating cancer-induced osteolytic disease in a subject. All of the embodiments/disclosures described herein in relation to the methods of treatment of cancer-induced osteolytic disease in a subject apply equally to this medical use aspect of the disclosure.

### Sclerostin Antagonists

Sclerostin is a naturally occurring protein that in humans is encoded by the SOST gene. Sclerostin is a secreted glycoprotein with a C terminal cysteine knot-like (CTCK) domain and sequence similarity to the DAN (differential screening-selected gene aberrative in neuroblastoma) family of bone morphogenetic protein (BMP) antagonists. Sclerostin is produced primarily by osteocytes but is also expressed in other tissues. The amino acid sequence of human sclerostin is provided in SEQ ID NO:1.

Sclerostin antagonists are known in the art, and include antibodies, small molecule compounds and oligonucleotides. Thus, in one embodiment, the sclerostin antagonist is an antibody, a small molecule compound or an oligonucleotide. Small molecule sclerostin antagonists are described in WO2014153203, for example, which are expressly incorporated herein by reference thereto. Examples of oligonucleotide sclerostin antagonists include siRNA and antisense oligonucleotides. Methods for identifying further sclerostin antagonists are known in the art, as described in EP2277522 and U.S. Pat. No. 9,791,462, for example, which methods are expressly incorporated herein by reference thereto.

The sclerostin antagonist of the invention typically binds human sclerostin with high affinity. As used herein, the term “high affinity” refers to a sclerostin antagonist that binds human sclerostin with a K_{D} of 1×10⁻⁸ M or less, 1×10 ⁻⁹ M or less, 1×10⁻¹⁰ M or less, 1×10⁻¹¹ M or less or 1×10⁻¹² M or less. In one embodiment, the sclerostin antagonist binds human sclerostin with a K_{D} of 1×10⁻⁸ M or less. In one embodiment, the sclerostin antagonist binds human sclerostin with a K_{D} of 1×10⁻⁹ M or less.

In one embodiment, the sclerostin antagonist binds human sclerostin with a K_{D} of 1×10⁻¹⁰ M or less. In one embodiment, the sclerostin antagonist binds human sclerostin with a K_{D} of 1×10⁻¹¹ M or less. In one embodiment, the sclerostin antagonist binds human sclerostin with a K_{D} of 1×10⁻¹² M or less. Higher affinity binding is generally preferred. Typically, the sclerostin antagonist binds human sclerostin with a K_{D} of 1×10⁻⁸ M to 1×10⁻¹⁰ M . The term “K_{D}”, as used herein, is intended to refer to the dissociation constant, which is obtained from the ratio of K_{d} to Kₐ (i.e. K_{d}/Kₐ) and is expressed as a molar concentration (M). K_{D} values can be determined using methods well established in the art. A method for determining the K_{D} of an antibody, for example, is by using surface plasmon resonance, or using a biosensor system such as a Biacore® system.

The sclerostin antagonist may also block the inhibitory effect of sclerostin in a cell based wnt signaling assay. In relation to a sclerostin antagonist that “blocks the inhibitory effect of sclerostin in a cell based wnt signaling assay”, in one embodiment this is intended to refer to a sclerostin antagonist that restores wilt induced signaling in the presence of sclerostin in a cell-based super top flash (STF) assay with an IC50 less than 1 mM, 100 nM, 20 nM, 10 nM or less. WO2009/047356 describes said wnt STF assay in more detail, which disclosure is expressly incorporated by reference thereto.

Anti-sclerostin antibodies represent preferred sclerostin antagonists of the invention, and suitable anti-sclerostin antibodies and methods of making anti-sclerostin antibodies are disclosed in WO2009047456, for example, which methods and antibodies are expressly incorporated into the present disclosure by reference thereto, expressly including the 6CDRs, VH and VL sequences as well as full length heavy and light chain sequences of these antibodies. In a preferred embodiment, the anti-sclerostin antibody is setrusumab, which is an anti-sclerostin monoclonal antibody. Setrusumab comprises the following CDRs: heavy chain variable region CDR1 of SEQ ID NO: 2; heavy chain variable region CDR2 of SEQ ID NO: 3; heavy chain variable region CDR3 of SEQ ID NO: 4; light chain variable region CDR1 of SEQ ID NO; 5; light chain variable region. CDR2 of SEQ ID NO: 6; and light chain variable region CDR3 of SEQ ID NO: 7. The VH and VL sequences of setrusumab comprise: the VH polypeptide amino acid sequence set forth as SEQ ID NO: 8 and the VL polypeptide amino acid sequence set forth as SEQ ID NO: 9. The heavy and light chain sequences of setrusumab comprise: the heavy chain polypeptide amino acid sequence set forth as SEQ ID NO: 10 and the light chain polypeptide amino acid sequence set forth as SEQ ID NO: 11.

Thus, in one embodiment, the anti-sclerostin antibody comprises at least one or more complementarity determining region (CDR) sequences selected from the group consisting of: (a) heavy chain variable region CDR1 comprising an amino acid sequence set forth in SEQ ID NO: 2; (b) heavy chain variable region. CDR2 comprising an amino acid sequence set forth in SEQ ID NO: 3; (c) heavy chain variable region CDR3 comprising an amino acid sequence set forth in SEQ ID NO: 4; (d) light chain variable region CDR1 comprising an amino acid sequence set forth in SEQ ID NO: 5; (e) light chain variable region CDR2 comprising an amino acid sequence set forth in SEQ ID NO: 6; and (f) light chain variable region CDR3 comprising an amino acid sequence set forth in SEQ ID NO: 7. In one embodiment, the anti-sclerostin antibody comprises at least the heavy chain variable region CDR3 comprising an amino acid sequence set forth in SEQ ID NO: 4. In one embodiment, the anti-sclerostin antibody comprises all 6 of the aforementioned CDRs.

In another embodiment, the anti-sclerostin antibody comprises:(a) a heavy chain variable region CDR1 comprising an amino acid sequence set forth in SEQ ID NO:2; (b) a heavy chain variable region CDR2 comprising an amino acid sequence set forth in SEQ ID NO:3; (c) a heavy chain variable region CDR3 comprising an amino acid sequence set forth in SEQ ID NO:4; (d) a light chain variable region CDR1 comprising an amino acid sequence set forth in SEQ ID NO:5; (e) a light chain variable region CDR2 comprising an amino acid sequence set forth in SEQ ID NO:6; and (f) a light chain variable region CDR3 comprising an amino acid sequence set forth in SEQ ID NO:7; or an anti-sclerostin antibody that binds to the same epitope as an anti-sclerostin antibody comprising: (a) a heavy chain variable region CDR1 comprising an amino acid sequence set forth in SEQ ID NO:2; (b) a heavy chain variable region CDR2 comprising an amino acid sequence set forth in SEQ ID NO:3; (c) a heavy chain variable region CDR3 comprising an amino acid sequence set forth in SEQ ID NO:4; (d) a light chain variable region CDR1 comprising an amino acid sequence set forth in SEQ ID NO:5; (e) a light chain variable region CDR2 comprising an amino acid sequence set forth in SEQ ID NO:6; and (f) a light chain variable region CDR3 comprising an amino acid sequence set forth in SEQ ID NO:7. In a related embodiment, the anti-sclerostin antibody binds to the same epitope as an anti-sclerostin antibody comprising a VL polypeptide sequence having the amino acid sequences set forth as SEQ ID NO:9 and a VH polypeptide sequence having a the amino acid sequences set forth SEQ ID NO:8. In another embodiment, the anti-sclerostin antibody binds to the same epitope as an anti-sclerostin antibody comprising a full length light chain amino acid sequence having the amino acid sequence set forth as SEQ ID NO:11 and a full length heavy chain amino acid sequence having the amino acid sequence set forth as SEQ ID NO:10. in one embodiment, the anti-sclerostin antibody binds to the same epitope as an anti-sclerostin antibody comprising a full length light chain amino acid sequence having the amino acid sequence set forth as SEQ ID NO:11 and a full length heavy chain amino acid sequence having the amino acid sequence set forth as SEQ ID NO:10.

In another embodiment, the anti-sclerostin antibody comprises a VH having at least 90 (such as at least 95, 98, or 99 or 100) percent identity (%) to the amino acid sequence set forth in SEQ ID NO: 8. In yet another embodiment, the anti-sclerostin antibody comprises a VL having at least 90 (such as at least 95, 98, or 99 or 100) percent identity (%) to the amino acid sequence set forth in SEQ ID NO: 9. In still another embodiment, the anti-sclerostin antibody comprises a VH having at least 90 (such as at least 95, 98, or 99 or 100) percent identity (%) to the amino acid sequence set forth in SEQ ID NO: 8, and a VL having at least 90 (such as at least 95, 98, or 99 or 100) percent identity (%) to the amino acid sequence set forth in SEQ ID NO: 9.

In yet another embodiment, the anti-sclerostin antibody comprises a VH having the amino acid sequence set forth in SEQ ID NO: 8, and a VL having the amino acid sequence set forth in SEQ ID NO: 9. In yet still another embodiment, the anti-sclerostin antibody comprises a heavy chain having at least 90 (such as at least 95, 98, or 99 or 100) percent identity (%) to the amino acid sequence set forth in SEQ ID NO: 10, and/or at least 90 (such as at least 95, 98, or 99 or 100) percent identity (%) to a light chain having the amino acid sequence set forth in SEQ ID NO: 11. In one embodiment, the anti-sclerostin antibody comprises a heavy chain having the amino acid sequence set forth in SEQ ID NO: 10 and a light chain having the amino acid sequence set forth in SEQ ID NO: 11.

Numerous examples of other anti-sclerostin antibodies and methods of making anti-sclerostin antibodies can be found in WO00/32773, WO2005/003158, WO2005/014650, WO2006/119107, WO2006/119062, WO2009/039175, WO2008/061013, and WO2008/115732. The methods and each of the anti-sclerostin antibodies disclosed in the aforementioned PCT publications and are expressly incorporated into the present disclosure by reference thereto, expressly including the 6CDRs, heavy chain variable sequences (VH), light chain variable sequences (VL) as well as full length heavy and light chain sequences of these antibodies.

In one embodiment, the anti-sclerostin antibody is romosozumab. Romosozumab comprises the full length heavy chain sequence represented by SEQ ID NO: 12 and the full length light chain sequence represented by SEQ ID NO: 13. Accordingly, in one embodiment the anti-sclerostin antibody comprises a full length heavy chain amino acid sequence comprising the amino acid sequence set forth as SEQ ID NO: 12 and a full length light chain amino acid sequence comprising the amino acid sequence set forth as SEQ ID NO: 13. In one embodiment, the anti-sclerostin antibody binds to the same epitope as romosozumab, that is, the anti-sclerostin antibody binds to the same epitope as an anti-sclerostin antibody comprising a full length heavy chain amino acid sequence having the amino acid sequence set forth as SEQ ID NO: 12 and a full length light chain amino acid sequence having the amino acid sequence set forth as SEQ ID NO: 13.

In another embodiment, the anti-sclerostin antibody is blosozumab. Blosozumab comprises the full length heavy chain sequence represented by SEQ ID NO: 14 and the full length light chain sequence represented by SEQ ID NO: 15. Accordingly, in one embodiment the anti-sclerostin antibody comprises a full length heavy chain amino acid sequence comprising the amino acid sequence set forth as SEQ ID NO: 14 and a full length light chain amino acid sequence comprising the amino acid sequence set forth as SEQ ID NO: 15. In one embodiment, the anti-sclerostin antibody binds to the same epitope as blosozumab, that is, the anti-sclerostin antibody binds to the same epitope as an anti-sclerostin antibody comprising a full length heavy chain amino acid sequence having the amino acid sequence set forth as SEQ ID NO: 14 and a full length light chain amino acid sequence having the amino acid sequence set forth as SEQ ID NO: 15.

Anti-sclerostin antibodies are known in the art that bind to/within the following human sclerostin sequences: CGPARLLPNAIGRGKWWRPSGPDFRC (the “loop 2 epitope”; SEQ ID NO: 16) and/or DVSEYCRELHFTR SAKPVTELVCSGQCGPAR WWRPSGPDFRCIPDRYR LVASCKCKRLTR (the “T20.6 epitope” SEQ ID NO: 17), as exemplified in WO00/32773, WO2005/003158, WO2005/014650, WO2006/119107, WO2006/119062, WO2009/039175 and WO2008/061013, for example, and these are expressly incorporated into the present disclosure by reference thereto, as noted above. Thus, in one embodiment, the anti-sclerostin antibody of the invention binds to/within the loop 2 epitope” (SEQ ID NO: 16) and/or the “T20.6 epitope” (SEQ ID NO: 17).

### Anti-Sclerostin Antibodies

The term “antibody” as referred to herein includes whole antibodies and any antigen binding fragment (i.e., “antigen-binding portion”) or single chains thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The structures and locations of immunoglobulin variable domains, e.g., CDRs, may be defined using well known numbering schemes, e.g., the Kabat numbering scheme (Kabat, E. A., et al., 1991 Sequences of Proteins of Immunological Interest, Fifth Edition, U.S. Department of Health and Human Services, NIH Publication No. 91-3242) or the Chothia numbering scheme (Al Lazikani et al., (1997) J. Mol. Bio. 273:927 948). In one embodiment, the CDR regions are defined using the Kabat system. In another embodiment, the CDR regions are defined using the Chothia system.

In one embodiment, reference to an antibody herein embraces isolated, monoclonal or polyclonal antibodies. The anti-sclerostin antibody may be a human, humanized, mouse or chimeric antibody. The anti-sclerostin antibody may be a bispecific, multispecific, or single-chain antibody. In one embodiment, the anti-sclerostin antibody is a monoclonal antibody. In one embodiment, the anti-sclerostin antibody is a humanized antibody.

The term “antigen-binding portion” of an antibody (or simply “antigen portion”), as used herein, refers to full length or one or more fragments of an antibody that retain the ability to specifically bind to an antigen (e.g., sclerostin). It has been shown that the antigen-binding function of an antibody can be performed by fragments of a full-length antibody. Examples of binding fragments encompassed within the term “antigen-binding portion” of an antibody include a Fab fragment, a monovalent fragment consisting of the VL, VH, CL and CH1 domains; a Fab′ fragment, comprising a Fab fragment wherein the CH1 domain is extended by further amino acids, for example to provide a hinge region or a portion of a hinge region domain, a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989 Nature 341:544-546), which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., U.S. Pat. No. 5,892,019). Such single chain antibodies are also intended to be encompassed within the term antibody. These antibody fragments are obtained using conventional techniques known to those of skill in the art, and the fragments are screened for utility in the same manner as are intact antibodies. An antigen-binding fragment, variant, or derivative of an anti-sclerostin antibody of the invention includes, but is not limited to, a Fab, Fab′ and F(ab′)2, Fd, Fv, dAb, single-chain Fv (scFv), or disulfide-linked Fv (sdFv). In one embodiment, the anti-sclerostin antibody is a Fab, Fab′, F(ab′)2, Fd, dAb, Fv, single-chain Fv (scFv), or a disulfide-linked Fvs (sdFv).

In one embodiment, the anti-sclerostin antibody is of the IgM, IgE, or IgG isotype, such as the IgG1, IgG2, IgG3 or IgG4 isotype. As used herein, “isotype” refers to the antibody class that is provided by the heavy chain constant region genes. The heavy chain constant region can be an IgG1, IgG2, IgG3, IgG4, IgA, IgE, IgM or IgD constant region. In one embodiment, the heavy chain constant region is selected among the IgG2 and IgG4 isotypes.

An “isolated antibody”, as used herein, refers to an antibody that is substantially free of other antibodies having different antigenic specificities (e.g., an isolated antibody that specifically binds sclerostin is substantially free of antibodies that specifically bind antigens other than sclerostin). An isolated antibody that specifically binds sclerostin may, however, have cross-reactivity to other antigens, such as sclerostin molecules from other species. Moreover, an isolated antibody may be substantially free of other cellular material and/or chemicals. In one embodiment, reference to an antibody herein means an isolated antibody.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from sequences of human origin.

Furthermore, if the antibody contains a constant region, the constant region also is derived from such human sequences, e.g., human germline sequences, or mutated versions of human germline sequences or antibody containing consensus framework sequences derived from human framework sequences analysis as described in Knappik, et al. (2000, J Mol Biol 296, 57-86),

The human antibodies may include amino acid residues not encoded by human sequences (e.g., mutations introduced by random or site-specific mutagenesis in vitro or by somatic mutation in vivo). However, the term “human antibody”, as used herein, is not intended to include antibodies in which. CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectotria, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Standard assays to evaluate the binding ability of the antibodies toward sclerostin are known in the art, including for example, ELISAs, western blots and RIAs. Suitable assays are described in detail in WO2009/047356. The binding kinetics (e.g., binding affinity) of the antibodies also can be assessed by standard assays known in the art, such as by Biacore analysis. Assays to evaluate the effects of the antibodies on functional properties of sclerostin (e.g., receptor binding, preventing or ameliorating osteolysis) are described in further detail in. WO2009/047356. Thus, for example, an antibody that binds the same epitope as an anti-sclerostin antibody described herein can be identified by its ability to cross-block or be cross-blocked (e.g. competitively inhibit the binding of) with an anti-sclerostin antibody described herein in standard sclerostin binding assays. The ability of a test antibody to inhibit the binding of antibodies of the present invention to human sclerostin demonstrates that the test antibody can compete with that antibody for binding to human sclerostin; such an antibody may, according to non-limiting theory, bind to the same or a related (e.g., a structurally similar or spatially proximal) epitope on human sclerostin as the antibody with which it competes. The ability or extent to which an antibody or other binding agent is able to interfere with the binding of another antibody or binding molecule to human sclerostin, and therefore whether it can be said to cross-block or be cross-blocked, can be determined using standard competition binding assays. One suitable assay involves the use of the Biacore technology, which can measure the extent of interactions using surface plasmon resonance technology. Another assay for measuring cross-blocking uses an ELISA-based approach. Further details on these methods can be found in WO2009/047356, for example, which disclosure is expressly incorporated herein by reference thereto.

Additional characteristics of the anti-sclerostin antibody, such as setrusumab are described in WO2009/047356, which disclosure, discussion and data is hereby incorporated by reference thereto. By way of example only, the anti-sclerostin antibody may exhibit at least one of the following functional properties: the antibody blocks the inhibitory effect of sclerostin in a cell based mineralization assay, the antibody blocks the inhibitory effect of sclerostin in Smad1 phosphorylation assay, the antibody inhibits binding of sclerostin to the LRP-6, and the antibody increases bone formation and mass and density. As noted above, these properties are described in detail in WO2009/047356.

### Pharmaceutical Compositions

The sclerostin antagonists of the invention may also be formulated as part of a pharmaceutical composition comprising a sclerostin antagonist of the invention formulated together with a pharmaceutically acceptable carrier. Such compositions may include one or a combination of (e.g. two or more different) sclerostin antagonists of the invention. For example, a pharmaceutical composition of the invention can comprise two or more anti-sclerostin antibodies that bind to different epitopes on human sclerostin or that have otherwise complementary activities. The sclerostin antagonist of the invention and the additional therapeutic agent of the invention can also be formulated in a combined preparation.

The term “pharmaceutically acceptable carrier” includes any and all solvents, buffers, dispersion media, coatings, antibacterial and antifungal agents, isotonic and absorption delaying agents, and the like that are physiologically compatible. The carrier should be suitable for intravenous, intramuscular, subcutaneous, parenteral, spinal or epidermal administration (e.g., by injection or infusion). Depending on the route of administration, the sclerostin antagonist may be coated in a material to protect the compound from the action of acids and other natural conditions that may inactivate the compound. In some embodiments, a composition for intravenous administration typically is a solution in sterile isotonic aqueous buffer.

In some embodiments, pharmaceutically acceptable carriers include sterile aqueous solutions or dispersions and sterile powders for the extemporaneous preparation of sterile injectable solutions or dispersion. In certain embodiments, the disclosure provides a sterile powder of the sclerostin antagonist for the preparation of sterile injectable solutions. In the case of sterile powders for the preparation of sterile injectable solutions, the preferred methods of preparation are vacuum drying and freeze-drying (lyophilization) that yield a powder of the sclerostin antagonist plus any additional desired ingredient from a previously sterile-filtered solution thereof. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

### Dosing & Administration

The sclerostin antagonist of the invention can be administered by one or more routes of administration using one or more of a variety of methods known in the art. The skilled person will appreciate that the route and/or mode of administration will vary depending upon the antagonist in question and the desired results. Routes of administration include intravenous, intramuscular, intradermal, intraperitoneal, subcutaneous, spinal or other parenteral routes of administration, for example by injection or infusion. In one embodiment, administration occurs via the intravenous route. In one embodiment, administration occurs intravenously by way of an infusion. In another embodiment, administration occurs subcutaneously.

The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, the sclerostin antagonist can be administered by a nonparenteral route, such as a topical, epidermal or mucosal route of administration, for example, intranasally, orally, vaginally, rectally, sublingually or topically.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the sclerostin antagonist and the particular therapeutic effect to be achieved, and the limitations inherent in the art of compounding such an active compound for the treatment of sensitivity in individuals.

By way of example, dosage ranges from, about 0.0001 to 200 mg/kg, of the host body weight might be appropriate depending on the specific sclerostin antagonist selected. An exemplary treatment regime entails administration once per week, once every two weeks, once every three weeks, once every four weeks, once a month, once every 3 months or once every three to 6 months.

### General

The term “comprising” encompasses “including” as well as “consisting” e.g. a composition “comprising” X may consist exclusively of X or may include something additional e.g. X+Y.

The term “about” in relation to a numerical valuer is optional and means, for example, x±10%.

As used herein, the percent identity between two sequences is a function of the number of identical positions shared by the sequences (i.e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol, Biol. 48:444-453, 1970) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6.

### DESCRIPTION OF THE DRAWINGS

FIG. 1. Breast cancer-derived sclerostin inhibits osteoblast differentiation and cancer cell migration.

A) Sost mRNA expression was determined in non-metastatic MCF-7 and metastatic MDA-MB-231 breast cancer cells by qRT-PCR. B) Calvarial osteoblasts were differentiated into osteoblasts in the presence of control medium or cancer conditioned medium (CM) collected from MDA-MB-231 breast cancer cells. Osteoblast differentiation was determined by Alizarin Red staining. C) Calvarial osteoblasts were cultured in the presence of indicated concentration of MDA-MB-231-derived CM. Wnt signaling activity was determined by TopFlash reporter assay. D) Wnt signaling activity in calvarial osteoblasts cultured with control medium, CM from MDA-MB-231 cells transfected with negative control siRNA (siRNA neg CM) or siRNA against Sost (siRNA Sost CM). E) Quantification of a Transwell assay to determine M DA-MB-23 I breast cancer cell migration after transfection with negative control siRNA (siRNA neg CM) or siRNA against Sost (siRNA Sost CM). F) Calvarial cells were differentiated into osteoblasts in the control medium or cancer-conditioned medium (CM) collected from MDA-M B-231 metastatic breast cancer cells. Osteoblast differentiation was determined by quantification of Runx2 and osteocalcin (Oxn) gene expression. Data are represented as mean±SEM. Two-tailed Student's t-test was used to compare two groups A, D), and ANOVA followed by Tukey's post-hoc analysis was used to compare three or more groups C, E, F), *p<0.05, **p<0.01, ***p<0.001.

FIG. 2. Pharmacological inhibition of sclerostin reduces bone metastatic burden in mice.

MDA-MB-231 breast cancer cell stably expressing Luciferase gene were injected into the left ventricle of 8-week old female immunocompromised SCID mice. Micrometastases were detected two weeks after breast cancer cell injection by bioluminescence imaging (BLI) and mice were randomized and received wither vehicle or anti-sclerostin antibody (Scl-Ab, 100 mg/kg) once a week for four weeks. A+B) Tumor growth in bone was A) visualized and B) quantified by BLI. C, D) HLA protein and mRNA expression was determined by immunohistochemistry and qRT-PCR C) in the lung and D) in the brain. E, F) Quantification of the metastasis area in the tibia of cancer-bearing mice treated with vehicle (n=16 tibiae) or Scl-Ab (n =16 tibiae) using histological sections, Scale bar: 1 mm.

FIG. 3. Sclerostin antibody treatment protects froth breast cancer-induced bone destruction.

A, B) Microcomputed tomography (pCT) analysis of bone mass (BV/TV, Bone volume/total volume) in the A) femur and B) tibia of non-tumor-bearing mice and in mice with tumor treated with vehicle or anti-Sclerostin antibody (Scl-Ab). C) Tartrate-resistant acid phosphatase (TRAP) staining of the distal femur of tumor-bearing mice treated with vehicle or Scl-Ab. D) Histomorphometric analysis of the bone mass (BV/TV, bone volume/tissue volume) of the proximal tibia (healthy non-treated n=5, vehicle treated n=10, Scl-Ab treated n=10; cancer-bearing vehicle treated n=8, cancer-bearing Scl-Ab treated n=8). E) Analysis of the bone formation rate (BFR/BS, bone formation rate/bone surface) of the proximal tibia. F) Von. Kossa/van Gieson staining (two left panels) of the proximal tibia of mice with bone metastases and calcein double-labeling (two right panels) at the bone-cancer interface. Scale bars indicate 50 μm. G) Quantification of the bone formation rate per bone surface (BFR/BS) at the bone-cancer interface (vehicle n=6, Scl-Ab n=3). H) Immuno-histochemical staining of Osterix in the distal femur of cancer-bearing mice treated with vehicle or Scl-Ab. Scale bar indicates 50 μm. I)

Histomorphometric analysis of the distal femur (N.Ob/B.Pm, number of osteoblasts/bone perimeter; Ob.S/BS, osteoblast surface/bone surface) (vehicle n=6, Scl-Ab n=3). J) Tartrate-resistant acid phosphatase (TRAP) staining of the distal femur of cancer-bearing mice treated with vehicle or Scl-Ab. Scale bar indicates 50 μm. K) Quantification of the number of osteoclasts per bone perimeter (N.Oc/B.Pm) and of the osteoclast surface per bone surface (Oc.S/BS) (vehicle n=8, Scl-Ab n=8). Data are represented as mean±SEM. Two-tailed Student's t-test was used to compare two groups (A, B, G, I, K), and ANOVA followed by Tukey's post-hoc analysis was used to compare three or more groups (D, E), *p<0.05, **p<0.01, ***p<0.001. L) Von Kossa/van Gieson staining of the proximal tibiae and fluorescence double labeling (insets) from healthy mice and cancer-bearing mice treated with vehicle or Scl-Ab. Scale bars indicate 1 mm (black) and 50 μm (white).

FIG. 4. Inhibition of sclerostin prevents breast cancer-induced loss of muscle function and increases survival.

A) Force frequency of the EDL muscle of non-tumor-bearing mice treated with vehicle or anti-Sclerostin antibody (Scl-Ab). B) EDL muscle weight in non-tumor-bearing mice and mice with tumor treated with vehicle or Scl-Ab. C) Force frequency of the EDL muscle of non-tumor-bearing mice and mice with tumor treated with vehicle or Scl-Ab. D) TA muscle sections were stained with succinate staining and muscle fiber area was quantified using the Osteomeasure system. E) Survival curve of tumor bearing mice treated with vehicle or Scl-Ab. F) Specific force of the extensor digitorum longus (EDL) muscle from healthy mice without treatment (n=5), treated with vehicle (n=10) or anti-sclerostin antibody (Scl-Ab, n=10) and from cancer-bearing mice treated with vehicle (n=8) or Scl-Ab (n=8). G) Endurance of the EDL muscle of mice with bone metastases treated with vehicle (n=8) or Scl-Ab (n=8). H) Tibialis anterior muscle sections from healthy mice without treatment, vehicle or Scl-Ab treatment as well as from mice with bone metastases treated with vehicle or Scl-Ab stained for succinate dehydroxygenase (SDH). Two representative muscles are shown/group. Scale bar indicates 50 μm. I) Quantification of the cross-sectional area (CSA) of all muscle fibers, oxidative fibers (dark) and non-oxidative (light) fibers using SDH-stained muscle sections from healthy mice without treatment (n=5), vehicle (n=10) or Scl-Ab treatment (n=10) and from cancer-bearing mice treated with vehicle (n=8) or Scl-Ab (n=8). Data are represented as mean±SEM, Three or more groups were compared using ANOVA followed by Tukey's post-hoc analysis, *p<0.05, **p<0.01, ***p<0.001.

FIG. 5. Expression of sclerostin in primary breast cancer tissue from patients

SOST mRNA expression was analyzed in breast cancer tissue from 48 patients. Proportion of Sclerostin-positive and Sclerostin-negative tissue samples is shown in all patients and in triple-negative (ER-, PR-, HER-) and in hormone receptor-negative (ER-, PR-) patients. All, n=48; ER-, PR-, HER-, n=9; ER-, HER-, n=7.

FIG. 6. Molecular mechanisms underlying the effect of Scl-Ab on skeletal muscles of cancer-bearing mice

Treatment with an anti-sclerostin antibody restores breast cancer-induced activation of NF-κB signaling and increased number of Pax7-positive cells. A) Immunoblot analysis of phosphorylated IKKα and IKKβ (p-IKKα and p-IKKβ), phosphorylated NF-κBp65 (p-NF-κBp65), phosphorylated p38 (p-p38) and total p38 in the gastrocnemius (GAS) muscle of healthy non-treated mice (n=5) and cancer-bearing mice treated with vehicle (n=8) or Scl-Ab (n-8). Actin was used as loading control. Representative samples are shown. B) immunoblot analysis of phosphorylated NF-κBp65 (p-NF-κBp65), phosphorylated p38 (p-p38) and total p38 in C2C12 myoblasts stimulated with vehicle (veh) or TGF-β1. Actin was used as loading control. Representative image of 6 independent experiments is shown. C) Immunoblot analysis of phosphorylated NF-κBp65 (p-NF-κBp65), phosphorylated p38 (p-p38) and total p38 in C2C12 cells treated with a control peptide or an NF-κB blocking peptide (NPD) and stimulated with vehicle or TGF-β1. Actin was used as loading control. Representative image of 4 independent experiments is shown. D) Myogenin and MyoD mRNA expression was quantified by qRT-PCR in C2C12 cells after 10 days of myogenic differentiation (n=3). E) Pai1 mRNA expression was quantified in the GAS muscle from healthy non-treated mice (n=5) and from cancer-bearing mice treated with vehicle (n=8) or Scl-Ab (n=8). F) Immuno-histochemical staining of Pax7 in the TA muscle from healthy non-treated mice and from mice with bone metastases treated with vehicle or Scl-Ab. Scale bar indicates 50 μm in the upper panel and 100 μm in the lower panel. G) Quantification of Pax7-positive cells in the TA muscle from healthy non-treated mice (n=5) and from mice with bone metastases treated with vehicle (n=8) or Scl-Ab (n=8). Data are represented as mean±SEM. Two-tailed Student's t-test was used to compare two groups D), and ANOVA followed by Tukey's post-hoc analysis was used to compare three or more groups E, G), *p<0.05, ***p<0.001.

FIG. 7. LRP5 Mutations conferring resistance to breast cancer-mediated repression of Win signaling

Wnt signaling activity in calvarial osteoblasts isolated from mice heterozygous for the LRP5mutation G171V (LRP5-G171V+/T) and from control littermates (LRP5-G171V+/+) stimulated with control medium or cancer CM (n=4 independent experiments). ANOVA followed by Tukey's post-hoc analysis was used to compare three or more groups, *p<0.05, **p<0.01, ***p<0.001.

### MODES FOR CARRYING OUT THE INVENTION

### Example 1—Anti-Sclerostin Antagonists Treat Loss of Muscle Size, Mass, Strength and Function in a Mouse Tumour Model

### Methods

For calvarial osteoblast cultures, calvariae were dissected from 1-3-day old mice and digested sequentially in α-MEM containing 0.1% collagenase and 0.2% dispase. Cell fractions 2 to 4 were combined and expanded in α-MEM containing 10% FBS and P/S. C2C12 cell were purchased from. DSMZ and cultured in D-MEM (Invitrogen) containing 10% FBS and 1% Penicillin/streptomycin. Cells were stimulated with 10 ng/ml TGF-⊕1 (R&D) or 100 ng/ml recombinant sclerostin (R&D). To inhibit NF-kB signaling in. C2C12 cells, cells were pre-treated for 1 h with NEMO-binding domain peptide (NPD, Enzo) or L-TAT control peptide (Enzo). Myocyte differentiation was induced using D-MEM supplemented with 2% Horse Serum (Invitrogen) and 1% Penicillin/streptomycin. MCF-7 and MDA-MB-231 breast cancer cells were purchased from ATCC. MCF-7 cells were cultured in D-MEM (Invitrogen) and MDA-MB-231 cells were grown in α-MEM, both supplemented with 10% FBS and 1% Penicillin/streptomycin. All cell lines were tested for mycoplasma contamination. MDA-MB-231 cells were transfected with scrambled control siRNA or siRNA against sclerostin (Origene) using Lipofectamine 3000 (ThermoFischer) according to manufacturer's instructions. For collection of conditioned medium (CM), MDA-MB-231 cells were cultured in the presence of 1% FBS for 24 hours and CM was collected and stored at −80° C.

Osteoblast differentiation was induced by supplementing α-MEM with 0.2 mM L-ascorbic acid and 10 mM β-glycerophosphate. Osteoblast differentiation was determined by Alizarin Red staining after fixing the cells in 4% neutrally buffered formaldehyde solution. MCF-7 and MDA-MB-231 breast cancer cells were purchased from ATCC. MCF-7 cells were cultured in D-MEM and MDA-MB-231 cells in α-MEM, both supplemented with 10% Fetal bovine serum (FBS) and 1% Penicillin/streptomycin. RNA was isolated from cultured cells using the RN Easy kit and cDNA was synthesized using the NEB ProtoScript II First Strand cDNA Synthesis-kit according to manufacturer's instructions. Sclerostin/sost expression was determined by qRT-PCR using the following primers: Sost Forward: CCA CGG AAA TCA TCC CCG AG (SEQ ID NO: 18), Sost. Reverse: CAT CGG TCA CGT AGC GGG TG (SEQ ID NO: 19).

MDA-MB-231 cells were transfected with negative control siRNA or siRNA against Sost (purchased from Origene) using Lipofectamine. For collection of conditioned medium (CM) MDA-MB-231 cells were cultured in the presence of 1% FBS for 24 hours and conditioned medium was collected and stored at −80 C. Cell migration was determined using a Transwell assay. To determine Wnt signaling activity, calvarial osteoblasts were transfected with TopFlash and Renilla plasmids using a Neon System. Luciferase activity was measured using the Promega. Dual Luciferase-kit.

MDA-MB-231 breast cancer cells stably expressing Luciferase gene were injected into the left ventricle of 8-week old female immunocompromised SCID mice. Micrometastases were detected two weeks after injection of the breast cancer cells by bioluminescence imaging (BLI) and mice were randomized into two treatment arms. One group received vehicle (50 μl/10 g mouse) and the other one received the anti-sclerostin antibody sertrusumab (Scl-Ab) (100 mg/kg) intravenously (i.v) once a week for four weeks. Tumor burden was measured weekly by BLI. Mice were sacrificed one week after the last injection. In the survival study, mice were monitored daily and sacrificed once they reached well defined criteria such as 20% weight loss. Healthy CB-17/lcr-Prkdescid/Rj mice without treatment (n=10), treated with vehicle (n=10) or with Scl-Ab (n=10) served as control. Investigators were blinded to the group allocation.

Microcomputed tomography (μCT) was used for three dimensional analyses of long bones. Long bones of mice were analyzed using high-resolution microcomputed tomography with a fixed isotropic voxel size of 10 μm (70 peak kV at X μA 400 ms integration time; Viva80 micro-CT; Scanco Medical AG). This threshold was verified by manually evaluating 10 single tomographic slices from four samples per group to isolate the mineralized tissue and to preserve its morphology while excluding nonmineralized tissues. All analyses were performed on the digitally extracted bone tissue using 3D distance techniques (Scanco Medical AG). Region of interest (ROI) was defined manually by drawing contours in slices. Due to cancer-induced bone destruction and absence of intact bone surfaces in cancer-bearing mice, the ROI contained both the cortical and the trabecular bone. In non-cancer-bearing bones only trabecular bone was analyzed using a standard method (Bouxsein et al., J Bone Miner Res. 2013;28(1):2-17).

Ex vivo contractility of the Extensor digitorum longus muscle (EDL) was analyzed using a 1300A Whole Animal Muscle Test System (Aurora Scientific). For this purpose, EDL was dissected from the hind limb, hooks were tied to the tendons of the muscles and the muscles were mounted between a force transducer. The muscles were stimulated to contract using the supramaximal stimulus between two electrodes. For fatigue studies, EDL was stimulated with 70 Hz for 50 repeats and the maximum tetanic force was detected. Data were collected and analyzed using Dynamic Muscle Control/Data acquisition (DMC) and Dynamic Muscle Control Data Analysis (DMA) programs (Aurora Scientific). For this purpose, EDL was dissected from the hind limb, loops were tied to the tendons of the muscles and mounted to a force transducer. Muscles were stimulated to contract using a supramaximal stimulus applied by two electrodes. For fatigue studies, EDL was stimulated with 70 Hz for 50 repeats and the maximum tetanic force was detected. Data were collected and analyzed using Dynamic Muscle Control/Data acquisition (DMC) and Dynamic Muscle Control Data Analysis (DMA) programs (Aurora Scientific). Specific muscle force was calculated as described previously (Bonetto et al., Bonekey Rep. 2015;4:732). Investigators were blinded during data analysis.

For bone analyses, mice were injected seven and two days prior to sacrifice with calcein (20 mg/kg) and demeclocycline (20 mg/kg; both Sigma-Aldrich), respectively. Tibiae were collected and fixed in 4% paraformaldehyde (PFA) for 48 hours. For histomorphometric analysis, tibiae were embedded in methylmethacrylate. Toluidine blue, von Kossa/van Gieson and Tartrate-resistant acid phosphatase (TRAP) staining were performed using 5 μm sagittal sections. Quantitative bone histomorphometric measurements were performed according to standard protocols using an OsteoMeasure system (Osteometrics). Femura were cleaned from soft tissue, fixed in 4% PFA for 24 hours at +4° C., decalcified with 4% EDTA for 4 days and 20% EDTA for 24 hours and embedded in paraffin. Sections were cut and immuno-histochemical staining was performed using an anti-Osterix antibody (Rabbit polyclonal; Santa Cruz). Brain and lung tissues were fixed in 4% PFA for 24 hours at +4° C. Tissue samples were embedded in paraffin, cut and immuno-histochemical staining was performed with an antibody against HLA Class 1 ABC (Mouse monoclonal, Abeam). For histological analyses, the Tibialis anterior (TA) muscle was dissected from the hind limb, embedded in 10% Gum Tragacanth and snap-frozen in cooled 2-Methylbutane. Cryo sections were performed using a cryotome and sections were stained with succinate. For quantification of muscle fiber area (MFA), 12 μm thick cryo sections of the TA muscle were succinate stained (see reagents and protocol below) and fiber area and number were quantified using the Osteomeasure™ system (Osteometrics Inc., USA). Total fiber area was divided by the number of fibers to reach an average fiber area shown in FIG. 4B. MFA provides a measure of muscle fiber atrophy i.e. the size of individual muscles fibers. The investigators were blinded regarding the treatment.

### Succinate Staining Protocol

### Reagents:

0.2 M Sodium phosphate monobasic→sure at RT

02 M Sodium phosphate dibasic→4 store at RT

0.2 M Phosphate Buffer pH 7.6→store in +4° c.
13 ml 0.2 M sodium phosphate monobasic buffer
87 ml 0.2 M sodium phosphate dibasic buffer
Check pH and adjust to 7.6 using NaOH or HCL

0.2 M Sodium succinate solution→store at RT

NBT Solution→store in +4° c.
0.1 g NBT (Nitrotetrazolitun Blue chloride) Sigma #6876
50 ml dH2O

Incubation medium→prepare just before use.
10 ml 0.2 M phosphate buffer (mix on a heating plate 50-70° c.) until crystals are gone)
10 ml sodium succinate solution
10 ml NBT solution
10 ml dH2O

Physiological Saline→store at RT

Formalin-saline solution 9%-10%→prepare esh each time
90 ml physiological saline
10 ml 37%-40% formaldehyde

15% Ethanol

### Protocol:

Incubate sections for 30 min at 37° C. in incubation medium placed in a Coplin jar

Rinse sections in physiological saline

Fix sections in formal in saline solution (Coplin jar) for 3-5 in

Rinse in 15% Ethanol for 5 min in a Coplin jar

Mount with an aqueous mounting medium (aqua mount) or with glucerin gelatine and let stand for 2-3 min

Seal edges of cover slip with nail polish and let dry

Slides can be stored at RT

Femura were cleaned from soft tissue, fixed in 4% paraformaldehyde for 48 hours at +4 C, decalcified with EDTA and embedded in paraffin. Sections were cut and stained with Tartrate-resistant acid phosphatase (TRAP) staining (Taipaleenmaki et al. Oncotarget. 2016 Nov 29;¹7(48)^{.}39032-79046).

Lung and brain tissue were dissected from mice and cut in half. One half was snap frozen in liquid nitrogen and RNA was isolated using Trizol. RNA was isolated from cultured cells using the RNeasy Plus Mini-kit (Hagen). eDNA was synthesized using the NEB ProtoScript II First Strand cDNA Synthesis-kit and the expression of a human specific Human leukocyte antigen (HLA) gene, Myogenin, MyoD, Pai1 and SOST was analyzed by qRT-PCR. After normalization to GAPDH mRNA, relative expression level of each target gene was calculated using the comparative CT method.

The other half of the tissue was fixed in 4% paraformaldehyde for 24 hours at +4 C. Tissue samples were embedded in paraffin, cut and stained with an antibody against HLA (see Taipaleenmäki et al. Cancer Res, 2015 Apr. 1;75(7):1433-44).

Parametric data were analyzed using a two-tailed Student's t-test when two groups were compared. A one-way analysis of variance (ANOVA) was used when more than two groups were compared, followed by a Tukey's post-hoc analysis to compare the groups. Probability, values were considered statistically significant at p<0.05. Experiments were repeated at least three times as biological replicates with minimum two technical replicates. All quantitative data are represented as mean±SEM.

### Results

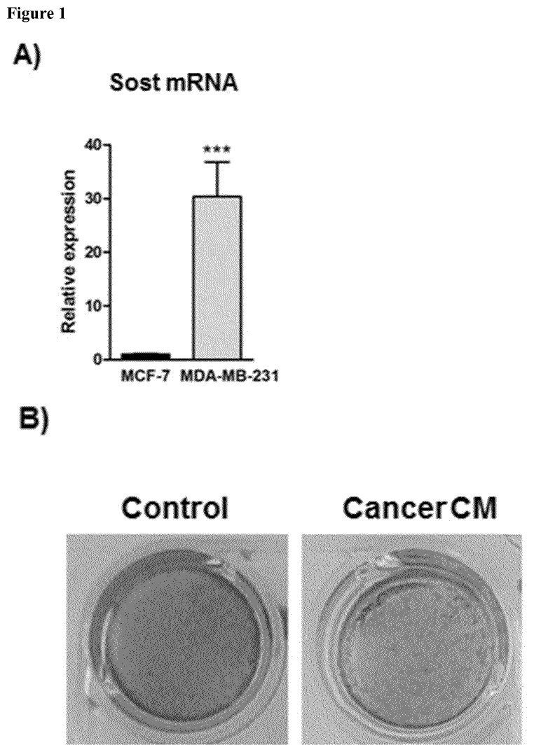

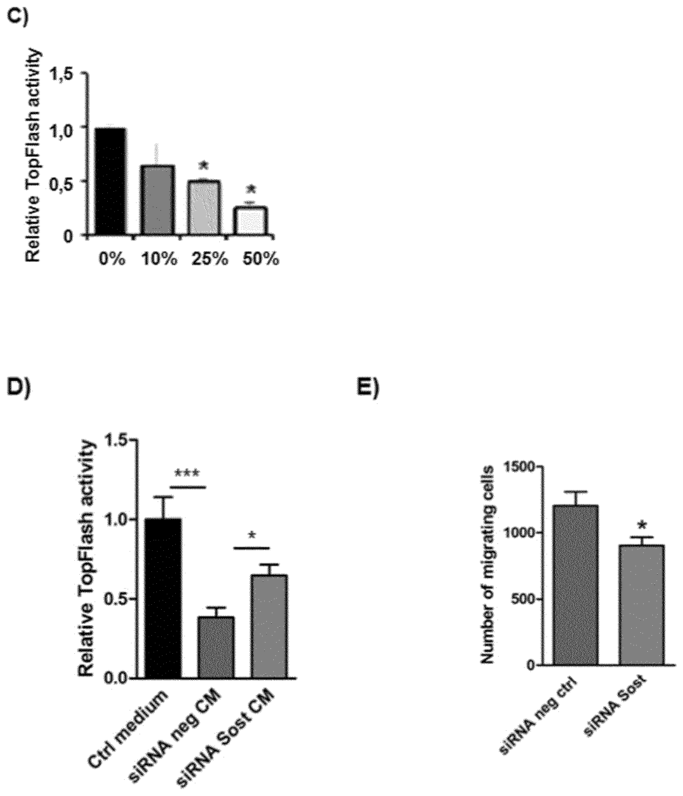

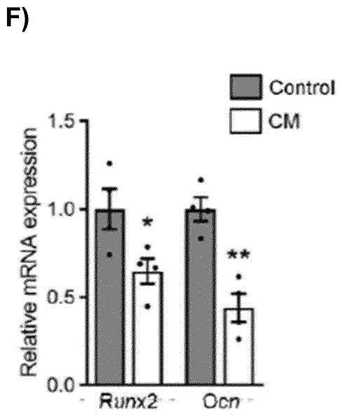

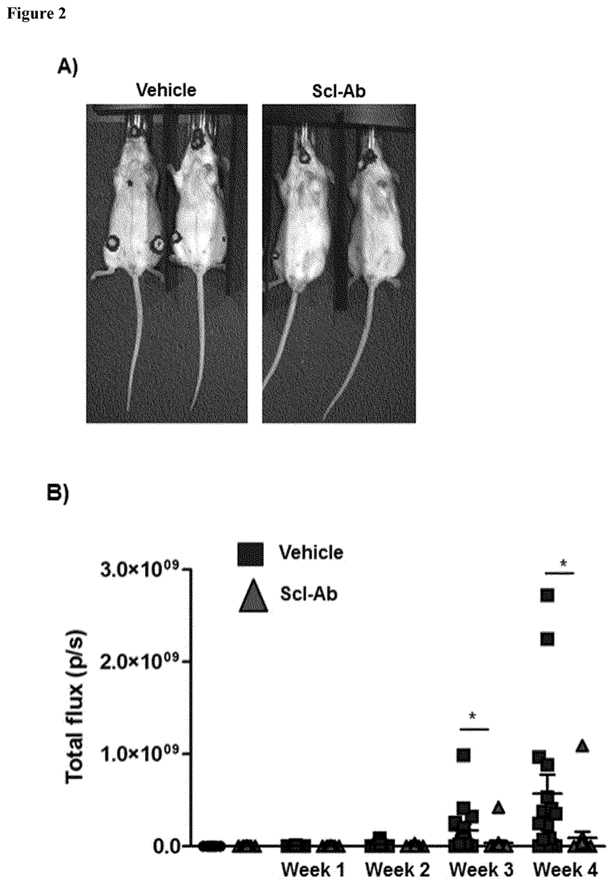

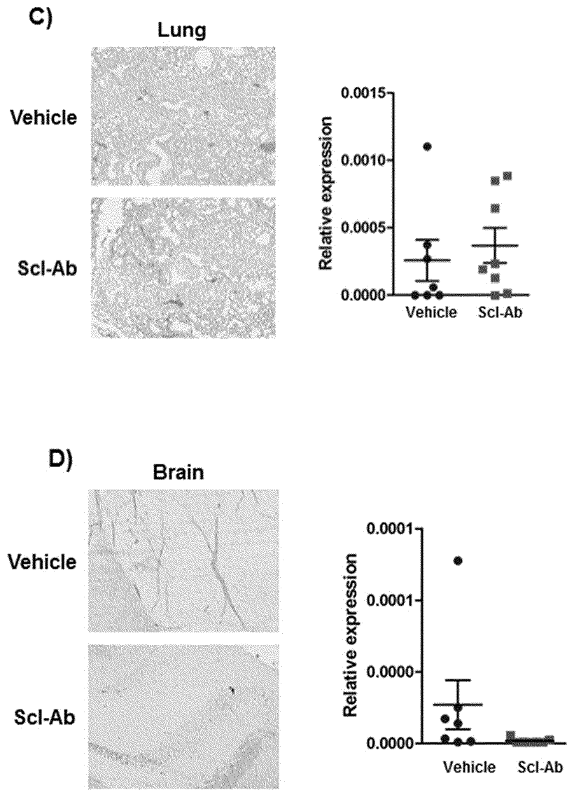

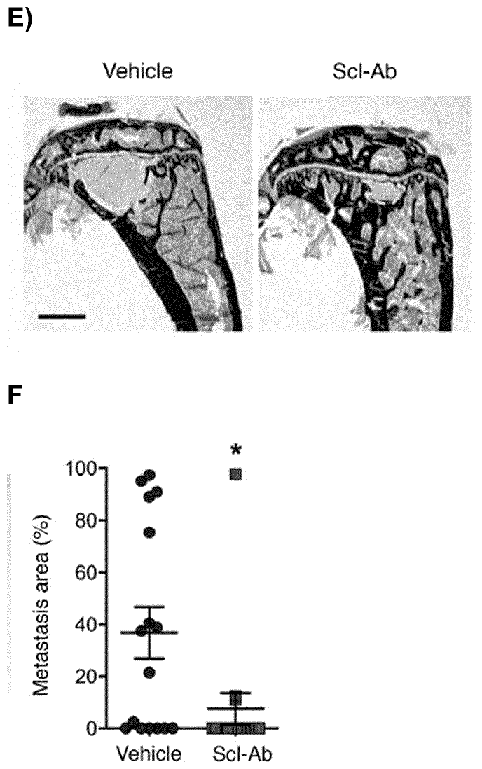

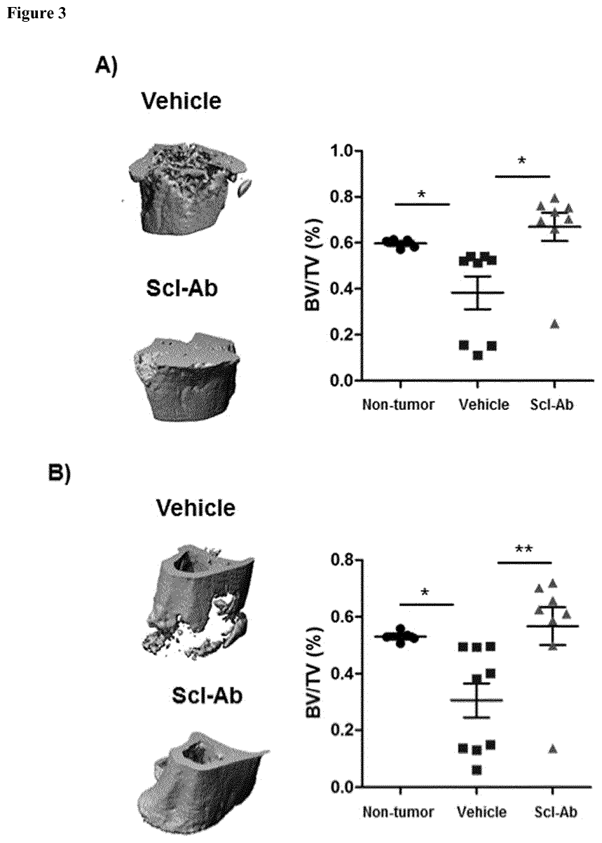

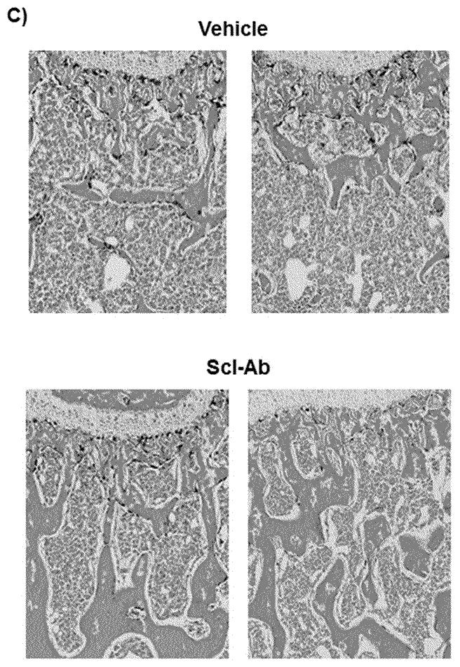

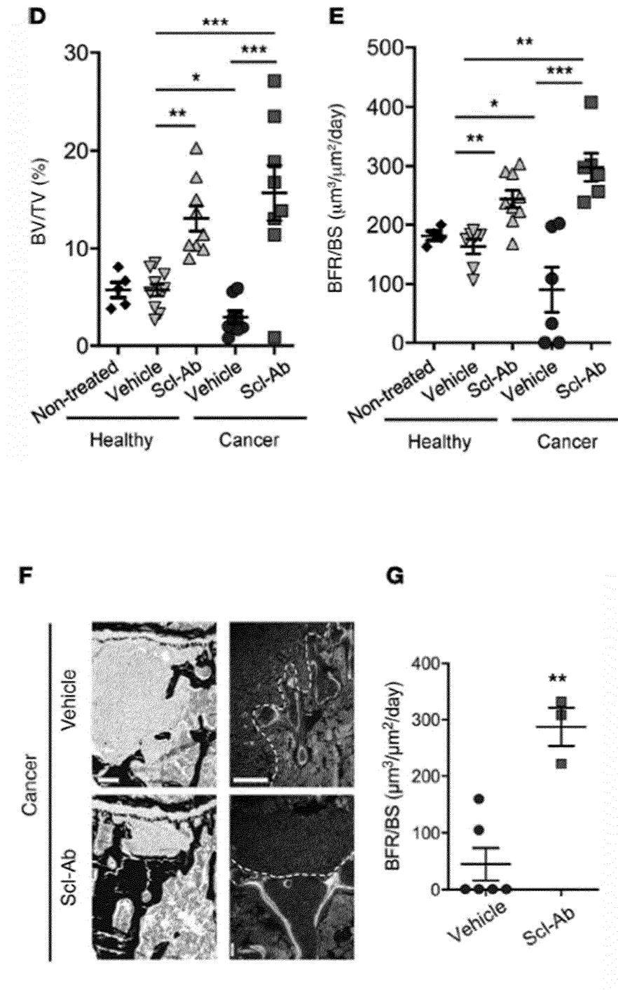

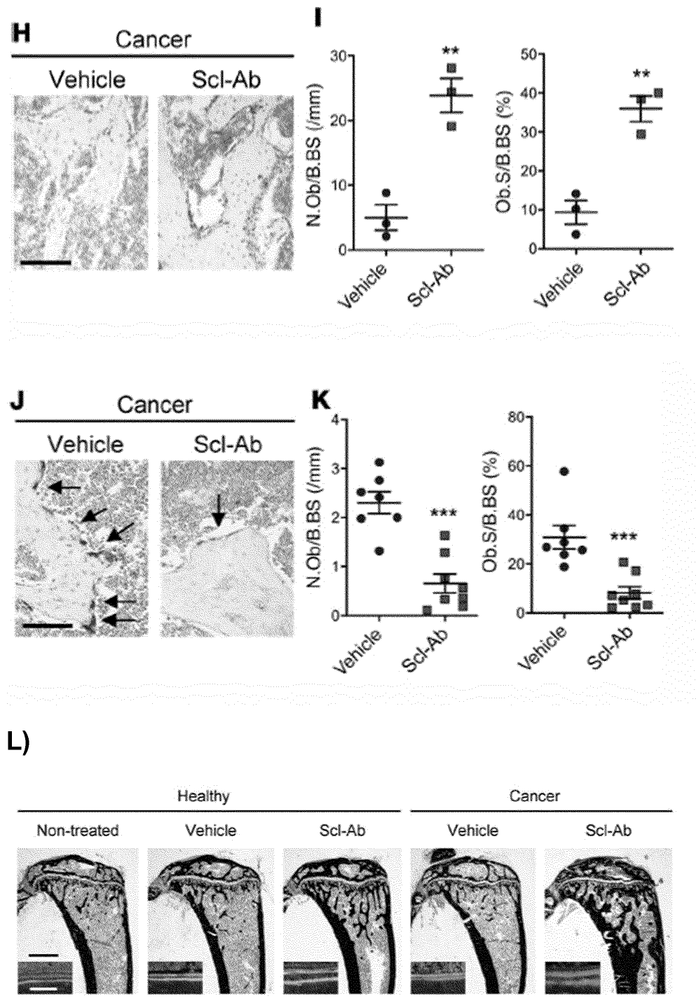

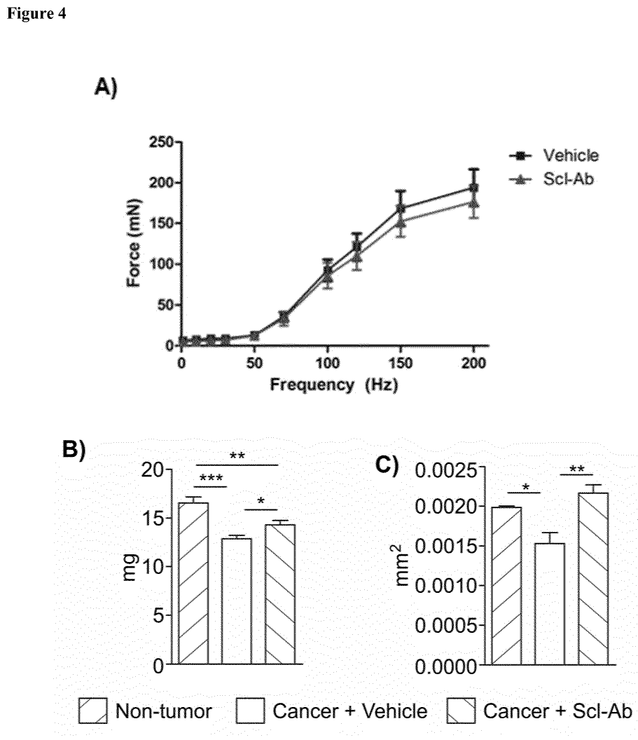

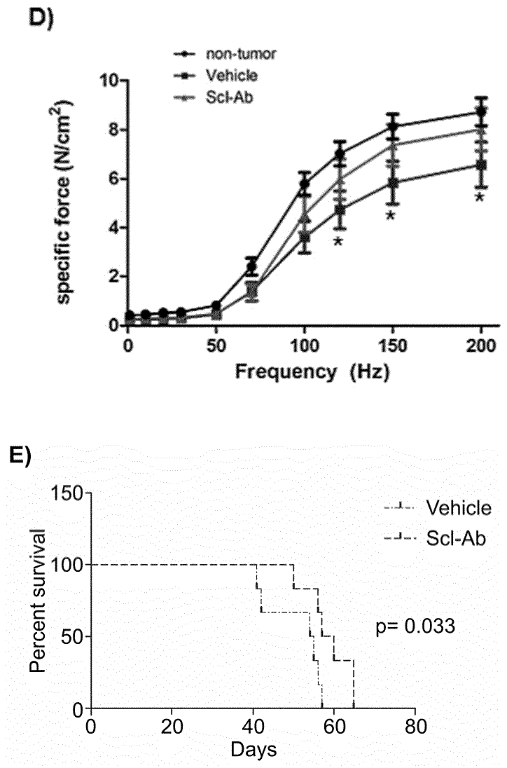

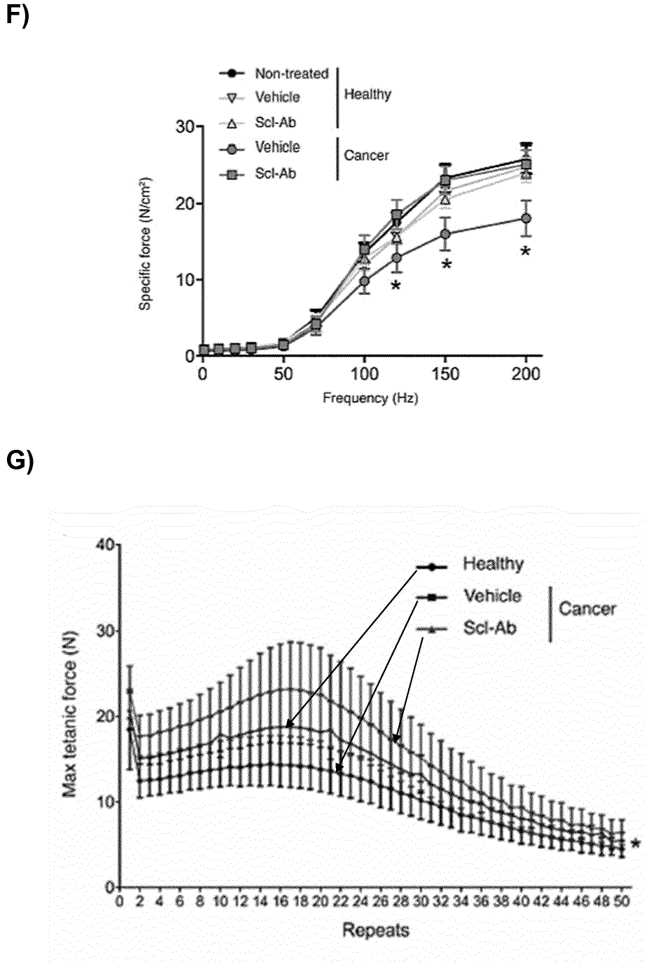

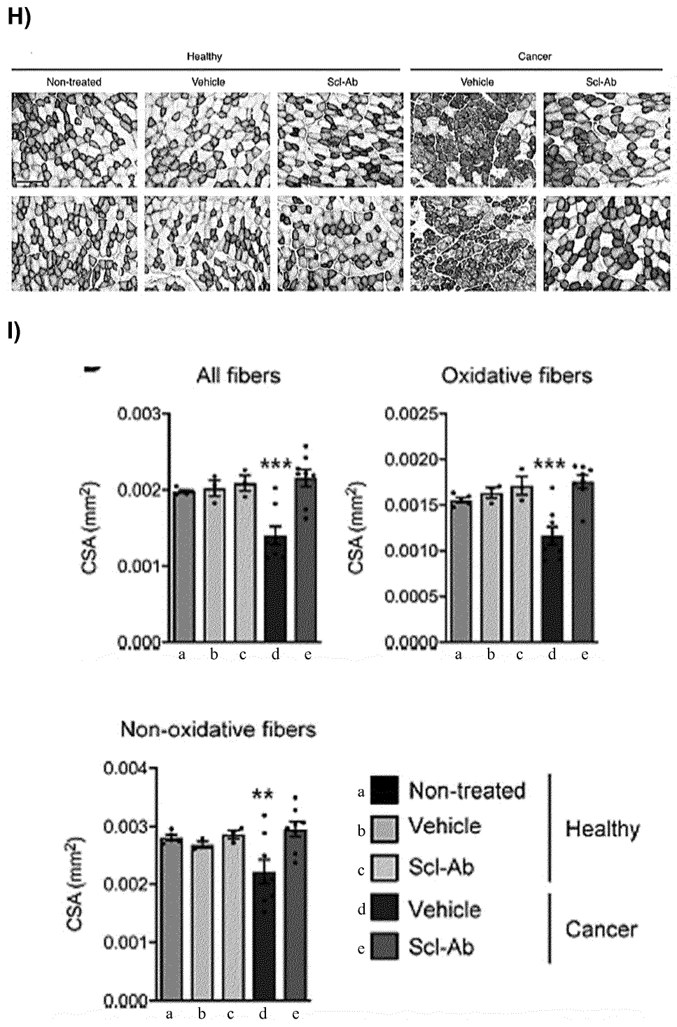

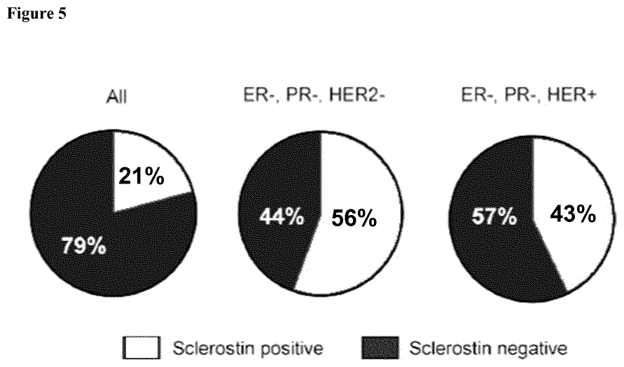

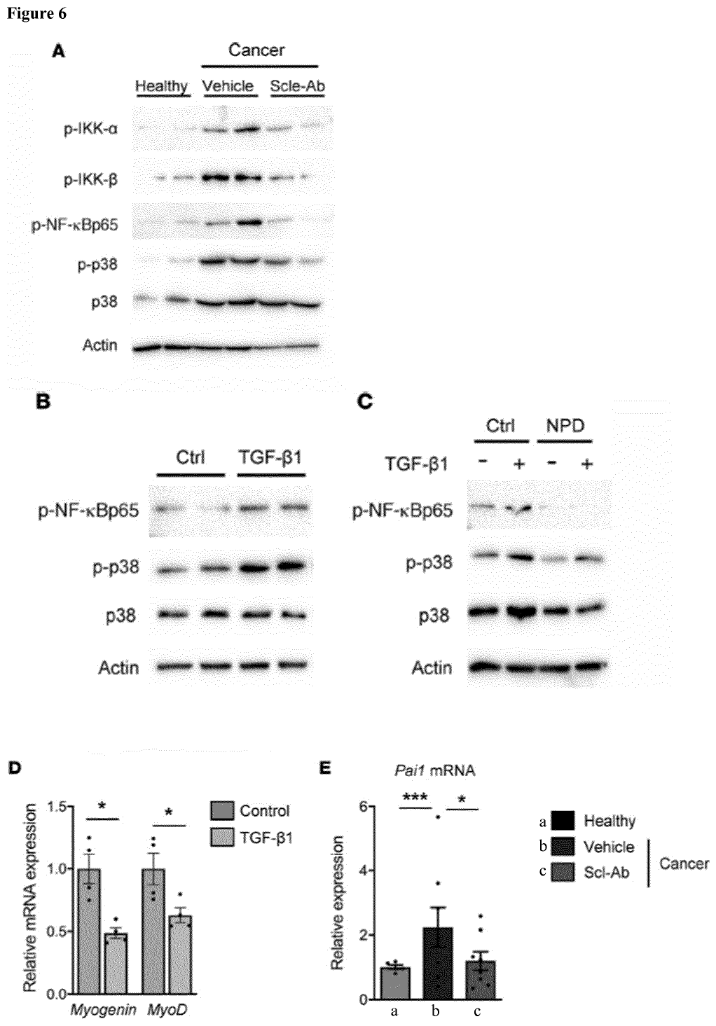

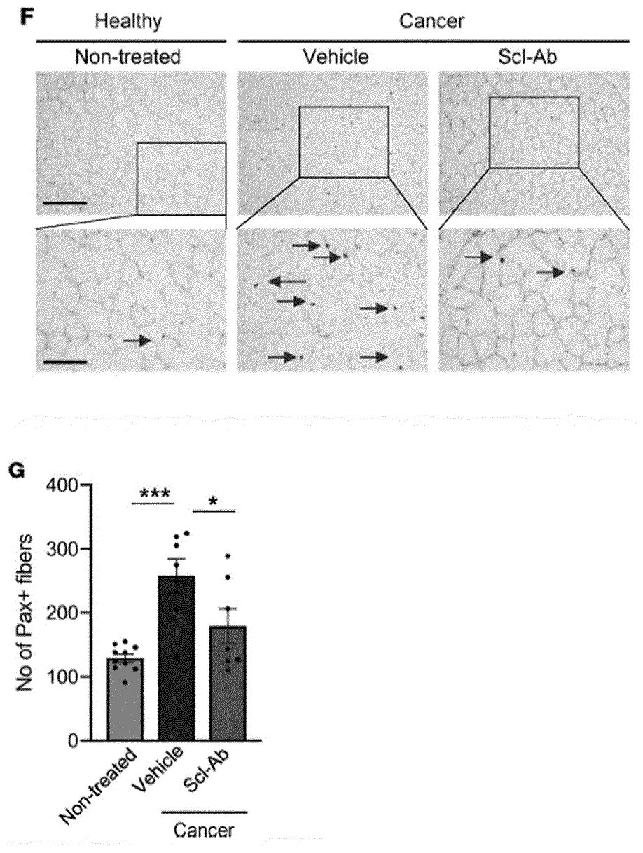

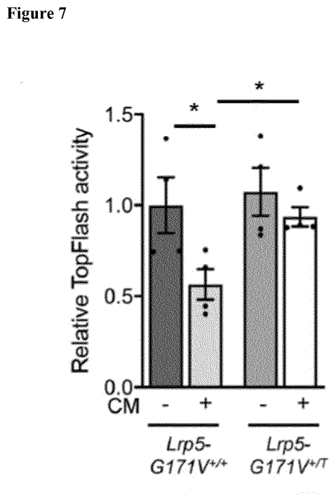

Sclerostin expression is significantly higher in metastatic MDA-MB-23I breast cancer cells compared to non-metastatic MCF-7 breast cancer cells (FIG. 1A). To address the hypothesis that metastatic breast cancer cells impair osteoblast differentiation, we collected conditioned medium from MDA-MB-231 metastatic breast cancer cells and differentiated primary calvarial cells into osteoblasts in the presence or absence of medium that had been conditioned by breast cancer cells. Osteoblast differentiation and the activity of Wnt signaling in osteoblasts was inhibited by conditioned medium (CM) from MDA-MB-231 breast cancer cells in a dose-dependent manner (FIGS. 1B, C). Conditioned medium inhibited osteoblast differentiation and matrix mineralization, demonstrated by a reduced expression of the osteoblast marker genes Runx2 and Ocn and a weaker Alizarin red staining (FIGS. 1F, B). These findings indicate that metastatic breast cancer cells may secrete factors that inhibit canonical Wnt signaling in osteoblasts in a paracrine fashion.

Repression of Wnt activity by CM was partially abolished upon antagonizing sclerostin in breast cancer cells using siRNA (FIG. 1D). Reduction of sclerostin expression in breast cancer cells also restricted cancer cell migration (FIG. 1E). This demonstrates that sclerostin derived from breast cancer cells impairs bone formation but supports metastatic features.

MDA-MB-231 breast cancer cells stably expressing the luciferase gene were delivered by cardiac injection in female SCID mice and metastases were allowed to form prior to treatment with vehicle or sertrusumab (Scl-Ab, 100 mg/kg). Non cancer-bearing mice without treatment, with vehicle or Scl-Ab treatment served as healthy controls. Weekly bioluminescence imaging revealed a reduced growth of bone metastases in Scl-Ab-treated mice compared to controls (FIGS. 2A, B). The reduced tumor growth was confirmed by histological analyses of the metastases area in the tibiae (FIGS. 2E, F) of cancer-bearing mice. To evaluate the potential effects of Scl-Ab treatment on cancer cell relocation and metastases formation at other sites, several organs including lung, liver and brain were imaged by ex vivo bioluminescence after sacrifice (data not shown). Inhibition of sclerostin did not change the abundance of breast cancer cells at extra-skeletal sites, indicating that breast cancer cells did not relocate to other organs (FIGS. 2C, D).

Scl-Ab treatment had no effect on muscle function in naïve mice (FIG. 4A), but it protected from cancer-induced reduction of muscle fiber area (FIG. 4B) and loss of muscle strength and function in tumor-bearing mice (FIG. 4C), and expanded the life span of these animals (FIG. 4D). In summary, the data demonstrate that pharmacological inhibition of sclerostin reduces bone metastatic burden, prevents cancer-induced osteolytic disease as well as the loss of skeletal muscle mass, size, strength and function.

### Example 2—Sclerostin Expression is a General Feature of Breast Cancer Cells

Breast cancer cells have been shown to express Dickkopf 1 (Dkk1), a soluble antagonist of canonical Wnt signaling. However, antagonizing cancer cell-derived Dkk1 did not fully restore the activity of the Wnt pathway, suggesting that additional mechanisms might exist. Indeed, expression analysis revealed a significantly higher expression of the Wnt inhibitor sclerostin in metastatic MDA-MB-231 breast cancer cells compared to non-metastatic MCF-7 breast cancer cells (FIG. 1A). To determine whether sclerostin expression is a general feature of breast cancer cells, an in silico analysis was performed using the EMBL-EBI Expression. Atlas (Papatheodorou I et al. Nucleic Acids Rs. 2018;46(D1):D246-D251). In addition to cells of the MDA-MB-231 cell line, expression of sclerostin was found in cells of the SUM159PT, CAL51, HCC 1187, HCC 1197, HCC 1395, HCC 1806 and KPL-4 breast cancer cell lines. Interestingly, six of these cell lines (SUM159PT, CAL51, HCC 1187, HCC 1197, HCC 1395 and HCC 1806) neither express the estrogen receptor (ER) nor the progesterone receptor (PR) and do not bear an amplification of HER-2/Neu gene (referred to as triple-negative breast cancer cells). Furthermore, although KPL-4 cells have a HER-2/Neu amplification, they do not express the ER or the PR, suggesting that sclerostin expression is a common feature of hormone receptor-negative breast cancer cells.

To address the question whether sclerostin is expressed in primary breast cancer tissue from patients, the inventors quantified the sclerostin expression in tissue biopsies obtained from 48 breast cancer patients and from four healthy individuals. SOST expression in 48 human breast cancer tissues was analyzed using the TissueScan Breast Cancer Array III (Origene) according to the manufacturer's instructions. A TissueScan Cancer Survey Array 96 I (Origene) consisting of 96 samples from 72 tumor samples and 24 non-malignant tissue samples from 8 different primary organs was utilized to analyze the expression pattern of SOST in various malignant tissues. The expression of SOST was quantified using qRT-PCR. BETA-ACTIN (ACTB) was used an internal control.

While sclerostin expression was not detected in healthy breast tissue, 21% of primary breast cancers expressed sclerostin (FIG. 5). Interestingly, 56% of triple-negative breast cancer tissues and 43% of ER-negative and PR-negative breast cancer tissues expressed sclerostin (FIG. 5). Furthermore, 2 out of 3 (66%) metastatic breast cancers with unknown receptor status were positive for sclerostin expression. Tumors expressing either the ER or the PR or both receptors did not express sclerostin (data not shown). To determine if selerostin expression is a specific feature of breast cancer or if it is also expressed by other types of cancer, the inventors analyzed sclerostin expression in human colon (n=9), kidney (n=9), liver (n=9), lung (n=9), prostate (n=9), ovary (n=9) and thyroid (n=9) cancer biopsies and the respective healthy tissue. Sclerostin expression was detected in two colon (22%), one ovary (11%) and two lung (22%) cancer tissues, suggesting that sclerostin expression is not specific to breast cancer (Table 1).

TABLE 1
Sclerostin expression in different cancers

| | Total | Sclerostin- | Sclerostin- |
|---|---|---|---|
| Cancer type | number | positive | negative |
| Adenocarcinoma of colon | 9 | 2 (22%) | 7 (78%) |
| Carcinoma of kidney, renal cell | 9 | 0 | 9 (100%) |
| Carcinoma of liver (all) | 9 | 0 | 9 (100%) |
| Hepatocellular | 8 | 0 | 8 |
| Cholangiocarcinoma of liver | 1 | 0 | 1 |
| Carcinoma of lung (all) | 9 | 2 (22%) | 7 (78%) |
| Adenocarcinoma of lung | 2 | 0 | 2 |
| Squamous cell carcinoma | 5 | 1 | 4 |
| Non-small cell carcinoma | 2 | 1 | 1 |
| Ovary cancer (all) | 9 | 1 (11%) | 8 (89%) |
| Tumor of ovary, borderline | 2 | 0 | 2 |
| Adenocarcinoma of ovary | 7 | 1 | 6 |
| Prostate (all) | 9 | 0 | 9 (100%) |
| Hyperplasia of prostate | 6 | 0 | 6 |
| Prostatitis | 2 | 0 | 2 |
| Adenocarcinoma of prostate | 1 | 0 | 1 |
| Carcinoma of thyroid | 9 | 0 | 9 (100%) |

### Example 3—LRP5 Mutations Conferring Resistance to Breast Cancer-Mediated Repression of Wnt Signaling

Sclerostin inhibits the activation of the canonical. Wnt signaling pathway in osteoblasts by binding to the first β-propeller domain of the extracellular region of the LRP5 receptor. Heterozygous missense mutations (G171V and A214V) within this domain of LRP5 cause a high bone mass phenotype in patients and in mice due to a reduced binding of sclerostin. To determine whether cancer cell-derived sclerostin inhibits Wnt activity in osteoblasts through Lrp5, the inventors obtained osteoblasts from genetically modified mice heterozygous for the Lrp5 mutation G171V. In support of our hypothesis, osteoblasts bearing a mutant Lrp5 with a disabled sclerostin binding site were resistant to breast cancer-mediated repression of Wnt signaling (FIG. 7). These data suggest that breast cancer cells impair osteoblast differentiation, at least in part, by a sclerostin/Lrp5-mediated inhibition of the canonical Wnt signaling pathway.

### Example 4—Sclerostin-Induced Bone Destruction and Effects of Scl-Ab Treatment

In addition, sclerostin inhibition prevented cancer-induced bone destruction determined by μCT and histological analyses (FIGS. 3A-C). Histological analyses of the proximal tibia revealed a significantly reduced bone formation rate and bone mass in cancer-bearing vehicle-treated mice compared to healthy vehicle-treated control animals (FIGS. 3L, D, E). Intriguingly, Scl-Ab treatment of mice with bone metastases not only restored bone mass comparable to the bone mass of healthy vehicle-treated control animals, but also increased both bone mass and bone formation to the level of healthy Scl-Ab-treated mice (FIGS. 3, D and E). Detailed histomorphometric analysis of the bone surfaces nearby metastases revealed that the presence of breast cancer cells blunted bone formation in vehicle-treated mice, which was significantly restored by Scl-Ab treatment (FIGS. 3, F and G). These results suggest that Scl-Ab prevents bone loss in the context of bone metastases at least in part by restoring breast cancer-induced inhibition of bone formation at the bone-tumor interface.

To further analyze the mode of action of Sci-Ab in mice with breast cancer bone metastases, amino pro-peptide of type 1 collagen (P1NP) and Tartrate-resistant acid phosphatase (TRAP) 5b were measured in the serum of cancer-bearing mice as biomarkers for bone formation and bone resorption, respectively. Enzyme-linked immunosorbent assay (ELISA) was used to determine P1NP and TRAP concentration in the mouse serum. All procedures were performed according to the manufacturer's (immunodiagnostics systems) instructions.

While the P1NP serum concentration was higher in Scl-Ab-treated mice, the serum concentration of TRAP5b was decreased compared to vehicle-treated animals, suggesting that Scl-Ab treatment activates bone formation and reduces bone resorption. This dual mode of action has been consistently reported in the context of the treatment of postmenopausal osteoporosis. To further investigate this finding at a cellular level, parameters of osteoblasts and osteoclasts were quantified in cancer-bearing mice. As expected, the number and size of bone-forming osteoblasts was significantly increased in Scl-Ab-treated cancer-bearing mice compared to vehicle-treated control animals (FIGS. 3H, I).

Furthermore, the number and size of bone-resorbing osteoclasts was strongly reduced in response to Scl-Ab treatment (FIGS. 3J, K), suggesting that the increase in bone mass was due to a concomitant increase in bone formation and a reduction of bone resorption. Together, these data strongly indicate that Scl-Ab treatment does not only increase bone mass through its anabolic action by restoring the tumor-induced impairment of osteoblast function, but also reduces the breast cancer-mediated increase in osteoclast activity, thereby reverting osteolytic disease.

### Example 5—Effect of Scl-Ab on Muscle Function

Patients with bone metastases often suffer from muscle weakness. Similarly, mice with breast cancer-induced metastatic bone disease have a reduced ex vivo muscle contractility compared to healthy animals (FIG. 4F). Given the beneficial effect of Scl-Ab in reducing tumor burden and bone destruction, the inventors postulated that inhibition of sclerostin might also affect muscle function. To test this hypothesis, the inventors analyzed the specific muscle force and endurance of the extensor digitorum longus (EDL) muscles of healthy mice and of mice with bone metastases treated with Scl-Ab or vehicle control. Although Scl-Ab treatment increased bone mass in healthy mice (FIGS. 3L, D), muscle function was not altered in these animals (FIG. 4F). In contrast, Scl-Ab treatment of mice bearing bone metastases protected from cancer-induced muscle weakness, determined by quantification of specific muscle force (FIG. 4F) and endurance (FIG. 4G). To better understand the disease mechanism of the altered muscle function, the tibialis anterior (TA) muscles were stained for succinate dehydrogenase and the cross-sectional area (CSA) of oxidative and non-oxidative fibers was quantified.

Consistent with an unchanged muscle function, Scl-Ab treatment of healthy mice did not affect the CSA of neither oxidative nor non-oxidative muscle fibers (FIGS. 4H, I). Interestingly, bone metastases caused a significant reduction of the CSA, which was fully restored by Scl-Ab treatment (FIGS. 4H, I). Since the oxidative muscle fibers were affected in cancer-bearing mice (FIG. 4I), it appears to be likely that metastatic bone disease may cause an oxidative stress and muscle fiber atrophy in skeletal muscles, which is prevented by treatment with Scl-Ab.

### Example 6—Molecular Mechanisms Underlying the Effect of Scl-Ab on Skeletal Muscles of Cancer-Hearing Mice

To further elucidate the molecular mechanisms underlying the effect of Scl-Ab on skeletal muscles of cancer-bearing mice, the inventors investigated various signaling pathways involved in oxidative stress.

Cells were lysed in low salt lysis buffer (pH 7.6) containing 50 mM Tris base, 150 mM NaCl, 0.5% Nonindet P-40 (NP-40), 0.25% Sodium deoxycholate and complete protease and phosphatase inhibitors (Roche). Muscle tissue was homogenized in lysis buffer (20 mM Tris, pH 7.8, 137 mM NaCl, 2.7 mM KCl, 1 mM MgCl2, 1% Triton X-100, 10% glycerol, 1 mM EDTA, 1 mM dithiothreitol and protease and phosphatase inhibitors) using a dounce homogenizer. Lysates were separated on 12% polyacrylamide gels and subjected to immunoblot analysis. Immunoblots were incubated over night at 4° C. with primary antibodies listed in Table 2.

TABLE 2
Antibodies used in this study

| Antibody | Host animal | Dilution | Source | Catalog no. | Application |
|---|---|---|---|---|---|
| Pax7 | Mouse | 1:10 | DSHB | — | IHC |
| HLA | Mouse | 1:100 | Abcam | ab70328 | IHC |
| Osterix | Rabbit | 1:500 | Santa Cruz | Sc-22536-R | IHC |
| | | | Biotechnology | | |
| p38 | Rabbit | 1:500 | Cell Signaling | 8690 | IB |
| | | | Technology | | |
| Phospho-p38 | Rabbit | 1:500 | Cell Signaling | 4511 | IB |
| | | | Technology | | |
| Phospho-IKKα | Rabbit | 1:500 | Elabscience | E-AB-20902 | IB |
| Phospho-IKKβ | Rabbit | 1:500 | Etabscience | E-AB-20903 | IB |
| Phospho-NF-κB p65 | Rabbit | 1:500 | Abcam | ab28856 | IB |
| Actin | Mouse | 1:5000 | Millipore | MAB1501 | IB |
| p44/42 MAPK (Erk1/2) | Mouse | 1:1000 | Cell Signaling | 9107 | IB |
| | | | Technology | | |
| Phospho-p44/42 MAPK | Rabbit | 1:1000 | Cell Signaling | 4370 | IB |
| (Erk1/2)(Thr202/204) | | | Technology | | |
| STAT3 | Mouse | 1:500 | Cell Signaling | 9139 | IB |
| | | | Technology | | |
| Phospho-STAT3 | Rabbit | 1:500 | Cell Signaling | 9145 | IB |
| (Tyr705) | | | Technology | | |

Peroxidase-labeled anti-rabbit or anti-mouse secondary antibodies (1:10 000, Santa Cruz, Cat. No: W401B, 4V402B) were used to visualize bands using the Clarity Western ECL Substrate (BioRad). Images of the iminunoblots were acquired using the ChemiDoc imaging system and Image Lab software (Bio-Rad).

WEestern blot analysis revealed an increased phosphorylation of p38, ERK 1/2 and STAT3 in the gastrocnemius (GAS) muscle of cancer-bearing mice compared to healthy control animals (FIG. 6A). Interestingly, the cancer-induced phosphorylation of p38 was restored in the muscles of Scl-Ab-treated animals (FIG. 6A), Since activation of the NF-κB pathway has been shown to be a critical component of skeletal muscle atrophy, the inventors investigated whether NF-κB signaling is also implicated in breast cancer-induced bone metastases. Under steady-state conditions, NF-κB is sequestered in the cytoplasm through an interaction with members of IκB family of inhibitor protein termed IκBs (Inhibitor of κB). Upon activation, the IKK complex, which contains the two IκB kinases IKKα and IKKβ, phosphorylates the IκB proteins thereby targeting them to ubiquitination and proteasomal degradation. Interestingly, IKKα and IKKβ were strongly phosphorylated in the muscles of cancer-bearing mice treated with vehicle compared to healthy animals. Furthermore, phosphorylation of the NF-κB subunit p65 was strongly increased in cancer-bearing mice, indicating an activated NF-κB signaling. Phosphorylation of IKKα, IKKβ and p65 was greatly reduced by Scl-Ab treatment (FIG. 6A), suggesting that the presence of breast cancer bone metastases activates the p38-NF-κB signaling cascade, which is attenuated by the inhibition of sclerostin.

To determine whether sclerostin directly activates the p38-NF-κB signaling pathway in myoblasts, the inventors stimulated C2C12 myoblasts with recombinant sclerostin. Sclerostin treatment did not induce phosphorylation of p38 or the components of the NF-κB pathway (data not shown), suggesting that the pathway is activated indirectly by cytokines present in the metastatic micro-environment. TGF-β1 is an abundant growth factor released from the bone matrix during breast cancer-induced bone resorption. Interestingly, stimulation of undifferentiated C2C12 myoblasts with TGF-β1 activated the p38-NF-κB pathway, thus recapitulating the effect observed in the muscles of cancer-bearing mice (FIG. 6B). Inhibition of the NF-κB pathway abrogated the TGF-β1-induced phosphorylation of p38 (FIG. 6C), suggesting that the effect of TGF-β1 is at least in part mediated via NF-κB. Consistently, TGF-β1 stimulation inhibited the differentiation of C2C12 myoblasts into myocytes as determined by a reduced expression of Myogenin and MyoD (FIG. 6D). These data suggest that TGF-β1 released from the bone matrix during osteolytic bone resorption reduces muscle function, which is prevented by Scl-Ab treatment. Indeed, the expression of the TGF-β1 target gene Pai1 was significantly increased in muscles of bone metastases-bearing mice compared to healthy animals (FIG. 6E), However, Pai1 expression was not increased in the muscles of Scl-Ab-treated tumor-bearing mice (FIG. 6E), indicating that Scl-Ab treatment restores the breast cancer-induced activation of the TGF-β1 and p38-NF-κB pathway.

In colon cancer, NF-κB accumulation prevents the downregulation of Pax7, leading to a compromised muscle regeneration and impaired skeletal muscle function. To address the question whether this also occurs in mice with breast cancer bone metastases, the inventors analyzed the number of Pax7-positive satellite cells in the tibialis anterior muscles. Interestingly, the number of Pax7-positive cells was significantly increased in cancer-bearing mice compared to healthy animals (FIGS. 6, F and G). This cancer-induced increase was partially but significantly restored by Scl-Ab treatment (FIG. 6G). Together, these data suggest that pharmacological inhibition of sclerostin protects from breast cancer-induced loss of muscle function by preventing the cancer-mediated activation of NF-κB signaling and the subsequent increase of Pax7-positive satellite cells.

It will be understood that the invention has been described by way of example only and modifications may be made whilst remaining within the scope and spirit of the invention.

1. A method for treating a myopathy in a subject, comprising administering a therapeutically effective amount of a sclerostin antagonist to the subject.

2. The method according to claim 1, wherein the myopathy is characterized by a loss of skeletal muscle mass, size, strength and/or function.

3. The method according to claim 1 or claim 2, wherein the myopathy is cachexia, sarcopenia, or muscular dystrophy (MD) such as Duchenne muscular dystrophy (DMD) or Becker muscular dystrophy (DMB).

4. The method according to claim 1 or claim 2, wherein the myopathy is a cancer-associated loss of skeletal muscle mass, size, strength and/or function, such as cancer cachexia, cancer-associated myositis (CAM), inflammatory myopathy or steroid-induced loss of skeletal muscle mass, size, strength and/or function.

5. The method according to claim 4, wherein the cancer is breast cancer, lung cancer, prostate cancer, multiple myelcoma, cholangiocarcinoma, or hepatocellular carcinoma.

6. The method according to claim 4 or claim 5, wherein the cancer is breast cancer.

7. The method according to any preceding claim, wherein the sclerostin antagonist is an anti-sclerostin antibody, a small molecule compound or an oligonucleotide.

8. The method according to claim 7, wherein the anti-sclerostin antibody is a mouse, chimeric humanized or human antibody.

9. The method according to claim 7 or claim 8, wherein the anti-sclerostin antibody is a monoclonal antibody.

10. The method according to any one of claims 7-9, wherein the anti-sclerostin antibody is a humanized antibody.

11. The method according to any one of claims 7-10, wherein the anti-sclerostin antibody is a Fab, Fab′, F(ab′)2, Fd, dAb, Fv, single-chain Fv (scFv), or a disulfide-linked Fvs (sdFv).

12. The method according to any one of claims 7-11, wherein the anti-sclerostin antibody is of the IgG isotype, such as the IgG2 or IgG4 isotype.

13. The method according to any one of claims 7-12, wherein the anti-sclerostin antibody comprises:
(a) heavy chain variable region CDR1 comprising an amino acid sequence set forth in SEQ ID NO:2;
(b) heavy chain variable region CDR2 comprising an amino acid sequence set forth in SEQ ID NO:3;
(c) heavy chain variable region CDR3 comprising an amino acid sequence set forth in SEQ ID NO:4;
(d) light chain variable region CDR1 comprising an amino acid sequence set forth in SEQ ID NO:5;
(e) light chain variable region CDR2 comprising an amino acid sequence set forth in SEQ ID NO:6; and
(f) light chain variable region CDR3 comprising an amino acid sequence set forth in SEQ ID NO:7.

14. The method according to any one of claims 7-13, wherein the anti-sclerostin antibody comprises:
a) a VH polypeptide sequence having at least 90 percent sequence identity to the amino acid sequences set forth as SEQ ID NO:8; and/or
b) a VL polypeptide sequence having at least 90 percent sequence identity to the amino acid sequences set forth as SEQ ID NO:9.

15. The method according to claim 14, wherein the anti-sclerostin antibody comprises a VH polypeptide sequence comprising the amino acid sequence set forth as SEQ ID NO:8 and a VL polypeptide sequence comprising the amino acid sequence set forth as SEQ ID NO:9.

16. The method according to any one of claims 7-15, wherein the anti-sclerostin antibody comprises:
a) a full length heavy chain amino acid sequence having at least 90 percent sequence identity to the amino acid sequence set forth as SEQ ID NO:10; and/or
b) a full length light chain amino acid sequence having at least 90 percent sequence identity to the amino acid sequence set forth as SEQ ID NO:11.

17. The method according to claim 16, wherein the anti-sclerostin antibody comprises a full length heavy chain amino acid sequence comprising the amino acid sequence set forth as SEQ ID NO: 10 and a full length light chain amino acid sequence comprising the amino acid sequence set forth as SEQ ID NO: 11.

18. The method according to any one of claims 7-12, wherein the anti-sclerostin antibody binds to a sequence selected from SEQ ID NO: 16 and/or SEQ ID NO: 17.

19. The method according to any one of claims 7-12, wherein the anti-sclerostin antibody binds to the same epitope as an anti-sclerostin antibody comprising a VH polypeptide sequence having the amino acid sequences set forth as SEQ ID NO:8 and a VL polypeptide sequence having a the amino acid sequences set forth SEQ ID NO:9.

20. The method according to any one of claim 7-12 or 18, wherein the anti-sclerostin antibody binds to the same epitope an anti-sclerostin antibody comprising a full length heavy chain amino acid sequence having the amino acid sequence set forth as SEQ ID NO: 12 and a full length light chain amino acid sequence having the amino acid sequence set forth as SEQ ID NO: 13

21. The method according to any one of claims 7-12, wherein the anti-sclerostin antibody binds to the same epitope as an anti-sclerostin antibody comprising a full length heavy chain amino acid sequence having the amino acid sequence set forth as SEQ ID NO: 14 and a full length light chain amino acid sequence having the amino acid sequence set forth as SEQ ID NO: 15.

22. The method according to any one of claims 7-21, wherein the anti-sclerostin antibody is setrusumab, romosozumab, or blosozumab.

23. The method according to any preceding claim, comprising administering to the subject a therapeutically effective amount of an additional therapeutic agent, optionally wherein the additional therapeutic agent is an anti-cancer drug and/or an agent for the treatment of a myopathy.

24. The method according to claim 23, wherein the additional therapeutic agent is selected from one or more of:
a chemotherapy agent, such as imatinib, lenalidomide, bortezomib, leuprorelin, abiraterone and pemetrexed, a monoclonal antibody such as rituximab, bevacizumab, trastuzumab, and cetuximab, bone sparing drugs such as bisphosphonates, zoledronic acid, denosumab, alendronate, etidronate, ibandronate, risedronate, teriparatide, abaloparatide and calcitriol.

25. An anti-sclerostin antagonist for use in the treatment of a myopathy in a subject, optionally wherein the myopathy is defined as per any one of claims 2-6, and/or wherein the scerlostin antagonist is defined as per any one of claims 7-22.
